SEQUENCE LISTING (3 sequences)

SEQ ID NO: 1 - protein, 843 aa, Artificial Sequence
  Human Full-length Glucosidase I with 6-His affinity tag added to the C-terminus
MARGERRRRAVPAEGVRTAERAARGGPGRRDGRGGGPRSTAGGVALAVVVLSLALGMSGRWVLAWYRARRAVTLHSAPPVLPADSSSPAVAPDLFWGTYRPHVYFGMKTRSPKPLLTGLMWAQQGTTPGTPKLRHTCEQGDGVGPYGWEFHDGLSFGRQHIQDGALRLTTEFVKRPGGQHGGDWSWRVTVEPQDSGTSALPLVSLFFYVVTDGKEVLLPEVGAKGQLKFISGHTSELGDFRFTLLPPTSPGDTAPKYGSYNVFWTSNPGLPLLTEMVKSRLNSWFQHRPPGAPPERYLGLPGSLKWEDRGPSGQGQGQFLIQQVTLKIPISIEFVFESGSAQAGGNQALPRLAGSLLTQALESHAEGFRERFEKTFQLKEKGLSSGEQVLGQAALSGLLGGIGYFYGQGLVLPDIGVEGSEQKVDPALFPPVPLFTAVPSRSFFPRGFLWDEGFHQLVVQRWDPSLTREALGHWLGLLNADGWIGREQILGDEARARVPPEFLVQRAVHANPPTLLLPVAHMLEVGDPDDLAFLRKALPRLHAWFSWLHQSQAGPLPLSYRWRGRDPALPTLLNPKTLPSGLDDYPRASHPSVTERHLDLRCWVALGARVLTRLAEHLGEAEVAAELGPLAASLEAAESLDELHWAPELGVFADFGNHTKAVQLKPRPPQGLVRVVGRPQPQLQYVDALGYVSLFPLLLRLLDPTSSRLGPLLDILADSRHLWSPFGLRSLAASSSFYGQRNSEHDPPYWRGAVWLNVNYLALGALHHYGHLEGPHQARAAKLHGELRANVVGNVWRQYQATGFLWEQYSDRDGRGMGCRPFHGWTSLVLLAMAEDYHHHHHH

SEQ ID NO: 2 - protein, 976 aa, Artificial Sequence
  Alpha subunit of full-length murine glucosidase II
MGILPSPGMPALLSLVSLLSVLLMGCVAETGAVDRSNFKTCDESSFCKRQRSIRPGLSPYRALLDTLQLGPDALTVHLIHEVTKVLLVLELQGLQKNMTRIRIDELEPRRPRYRVPDVLVADPPTARLSVSGRDDNSVELTVAEGPYKIILTAQPFRLDLLEDRSLLLSVNARGLMAFEHQRAPRVPFSDKVSLALGSVWDKIKNLFSRQESKDPAEGNGAQPEATPGDGDKPEETQEKAEKDEPGAWEETFKTHSDSKPYGPTSVGLDFSLPGMEHVYGIPEHADSLRLKVTEGGEPYRLYNLDVFQYELNNPMALYGSVPVLLAHSFHRDLGIFWLNAAETWVDISSNTAGKTLFGKMLDYLQGSGETPQTDIRWMSESGIIDVFLMLGPSVFDVFRQYASLTGTQALPPLFSLGYHQSRWNYRDEADVLEVDQGFDDHNMPCDVIWLDIEHADGKRYFTWDPTRFPQPLNMLEHLASKRRKLVAIVDPHIKVDSGYRVHEELRNHGLYVKTRDGSDYEGWCWPGSASYPDFTNPRMRAWWSNMFSFDNYEGSAPNLYVWNDMNEPSVFNGPEVTMLKDAVHYGGWEHRDIHNIYGLYVHMATADGLIQRSGGIERPFVLSRAFFSGSQRFGAVWTGDNTAEWDHLKISIPMCLSLALVGLSFCGADVGGFFKNPEPELLVRWYQMGAYQPFFRAHAHLDTGRREPWLLASQYQDAIRDALFQRYSLLPFWYTLFYQAHKEGFPVMRPLWVQYPEDMSTFSIEDQFMLGDALLIHPVSDAGAHGVQVYLPGQEEVWYDIQSYQKHHGPQTLYLPVTLSSIPVFQRGGTIVPRWMRVRRSSDCMKDDPITLFVALSPQGTAQGELFLDDGHTFNYQTRHEFLLRRFSFSGSTLVSSSADPKGHLETPIWIERVVIMGAGKPAAVVLQTKGSPESRLSFQHDPETSVLILRKPGVSVASDWSIHLRASWSHPQFEK

SEQ ID NO: 3 - protein, 542 aa, Artificial Sequence
  Beta subunit of full-length murine glucosidase II
MGILPSPGMPALLSLVSLLSVLLMGCVAETGAVEVKRPRGVSLSNHHFYEESKPFTCLDGTATIPFDQVNDDYCDCKDGSDEPGTAACPNGSFHCTNTGYKPLYILSSRVNDGVCDCCDGTDEYNSGTVCENTCREKGRKEKESLQQLAEVTREGFRLKKILIEEWKTAREEKQSKLLELQAGKKSLEDQVETLRAAKEEAERPEKEAKDQHRKLWEEQQAAAKARREQERAASAFQELDDNMDGMVSLAELQTHPELDTDGDGALSEEEAQALLSGDTQTDTTSFYDRVWAAIRDKYRSEVPPTDIPVPEETEPKEEKPPVLPPTEEEEEEEEEPEEEEEEEEEEEEAPPPLQPPQPPSPTEDEKMPPYDEETQAIIDAAQEARSKFEEVERSLKEMEESIRSLEQEISFDFGPSGEFAYLYSQCYELTTNEYVYRLCPFKLVSQKPKHGGSPTSLGTWGSWAGPDHDKFSAMKYEQGTGCWQGPNRSTTVRLLCGKETVVTSTTEPSRCEYLMELMTPAACPEPPPEAPSDGDKHHHHHH

(19) United States
(12) Patent Application Publication
(10) Pub. No.: US 2024/0174619 A1
(43) Pub. Date: May 30, 2024

(54) VALIOLAMINE DERIVATIVES AS GLUCOSIDASE INHIBITORS

(71) Applicant: Emergent Product Development Gaithersburg Inc., Gaithersburg, MD (US)

(72) Inventors: Anthony M. TRESTON, Gaithersburg, MD (US); Kelly Lyn WARFIELD, Gaithersburg, MD (US)

(73) Assignee: EMERGENT PRODUCT DEVELOPMENT GAITHERSBURG INC., Gaithersburg, MD (US)

(21) Appl. No.: 18/279,920

(22) PCT Filed: Mar. 2, 2022
(86) PCT No.: PCT/US2022/018519

§ 371 Date: Sep. 1, 2023

Related U.S. Application Data
(60) Provisional application No. 63/201,194, filed on Apr. 16, 2021.
(60) Provisional application No. 63/156,253, filed on Mar. 3, 2021.

(51) Int. Cl. C07D 239/26; A61K 31/136; A61K 31/27; A61K 31/4192; A61K 31/505; C07C 215/44; C07C 217/08; C07C 229/10; C07C 247/16; C07C 271/20; C07D 249/06; C07D 257/04

(52) U.S. Cl. CPC C07D 239/26; A61K 31/136; A61K 31/27; A61K 31/4192; A61K 31/505; C07C 215/44; C07C 217/08; C07C 229/10; C07C 247/16; C07C 271/20; C07D 249/06; C07D 257/04

(57) ABSTRACT

This disclosure is directed to N-substituted valiolamine compounds, their use as glycosidase inhibitors, and in methods of treating diseases or conditions in which glycosidase inhibition provides benefit.

### REFERENCE TO A SEQUENCE LISTING

The instant application contains a Sequence Listing which has been submitted in ASCII format via EFS-Web and is hereby incorporated by reference in its entirety. The Sequence Listing is being concurrently submitted via EFS-Web as an ASCII text file named 2479_2220001_Seqlisting_ST25.txt, was created on Apr. 5, 2021, and contains 20,775 bytes.

### FIELD

Embodiments of the present disclosure generally relate to aminocyclitols—for example, N-substituted valiolamine compounds—and their use as glycosidase inhibitors. The present disclosure further provides methods of treating conditions and diseases, for which glycosidase inhibition provides benefit.

### BACKGROUND

Valiolamine is an aminocyclitol (also called pseudoaminosugar) and is a natural product identified in the fermentation broth of Streptomyces hygroscopicus subsp limoneus. Certain valiolamine derivatives are capable of inhibiting α-glucosidase and are suitable as pharmaceutical agents for treating diabetes. Voglibose ((1S,2S,3R,4S,5S)-5-(1,3-dihydroxypropan-2-ylamino)-1-(hydroxymethyl)cyclohexane-1,2,3,4-tetraol), for example, is a derivative of valiolamine and is an alphα-glucosidase inhibitor used for lowering post-prandial blood glucose levels in people with diabetes mellitus. Voglibose delays the absorption of glucose thereby reducing the risk of macrovascular complications. Given the prevalence of diabetes and the need for treatment of other diseases and conditions that are responsive to inhibition of α-glucosidase, incuding, for example, certain viral diseases, there remains a need in the art for novel valiolamine derivatives.

### BRIEF SUMMARY OF THE DISCLOSURE

The present disclosure is directed to N-substituted valiolamine compounds and their use as glycosidase inhibitors in treating diseases or conditions. More specifically, the present disclosure provides a compound according to Formula (I):

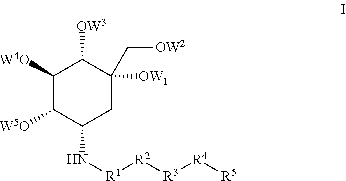

or a pharmaceutically acceptable salt thereof,
wherein
W¹-W⁵ are each independently selected from the group consisting of —H, —C(═O)—C₁-C₉ alkyl, and —C(═O)O—C₁-C₉ alkyl;
R¹ is optionally substituted C₁-C₉ alkylene;
R² is absent or selected from the group consisting of —H, —NH—, —O—, —C(═O)—, and —NH—C(═O)O—;
R³ is absent or selected from the group consisting of —O—, —C(═O)—, —C(═O)O—, and optionally substituted C₁-C₆ alkylene;
R⁴ is absent or selected from the group consisting of —H, —NH—, and optionally substituted C₁-C₆ alkylene;
R⁵ is absent, or is

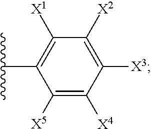

wherein
X¹, X², X³, X⁴, and X⁵ are each independently selected from the group consisting of —H, —NO₂, —N₃, optionally substituted C₂-C₁₂ heterocycle, and optionally substituted C₁-C₁₂ heteroaryl.

In some embodiments, W¹-W⁵ are each independently —H.

In some embodiments, R¹ is a C₁-C₉ alkylene.

In some embodiments, the C₁-C₉ alkylene is selected from the group consisting of —CH₂—, —CH₂—CH₂—, —(CH₂)₃—, —(CH₂)₄—, —(CH₂)₅—, —(CH₂)₆—, —(CH₂)₇—, —(CH₂)₈-, and —(CH₂)₉—.

In some embodiments, the C₁-C₉ alkylene is —(CH₂)₂—.

In some embodiments, the C₁-C₉ alkylene is —(CH₂)₄—.

In some embodiments, the C₁-C₉ alkylene is —(CH₂)₅—.

In some embodiments, the C₁-C₉ alkylene is —(CH₂)₆—.

In some embodiments, the C₁-C₉ alkylene is —(CH₂)₉—.

In some embodiments, R³ is a C₁-C₆ alkylene.

In some embodiments, the C₁-C₆ alkylene is selected from the group consisting of —CH₂—, —CH₂—CH₂—, —(CH₂)₃—, —(CH₂)₄—, —(CH₂)₅—, and —(CH₂)₆—.

In some embodiments, the C₁-C₆ alkylene is —CH₂—.

In some embodiments, the C₁-C₆ alkylene is —CH₂-CH₂—.

In some embodiments, R⁴ is a C₁-C₆ alkylene.

In some embodiments, the C₁-C₆ alkylene is selected from the group consisting of —CH₂—, —CH₂—CH₂—, —(CH₂)₃—, —(CH₂)₄—, —(CH₂)₅—, and —(CH₂)₆—.

In some embodiments, the C₁-C₆ alkylene is —CH₂—CH₂—.

In some embodiments, R⁵ is

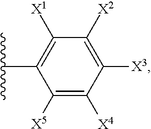

and X¹, X², X³, X⁴, and X⁵ are each independently selected from the group consisting of —H, —NO₂, —N₃, an optionally substituted C₂-C₁₂ heterocycle, and a C₁-C₁₂ heteroaryl.

In some embodiments, the C₁-C₁₂ heteroaryl is selected from the group consisting of

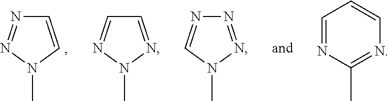

In some embodiments, X³ is —N₃ or a C₁-C₁₂ heteroaryl.

In some embodiments, X³ is —N₃.

In some embodiments, X³ is a C₁-C₁₂ heteroaryl, and the C₁-C₁₂ heteroaryl is selected from the group consisting of

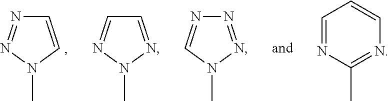

In some embodiments, X¹ is —NO₂.

In some embodiments, X², X⁴, and X⁵ are each —H.

In some embodiments, the compound is selected from the group consisting of:

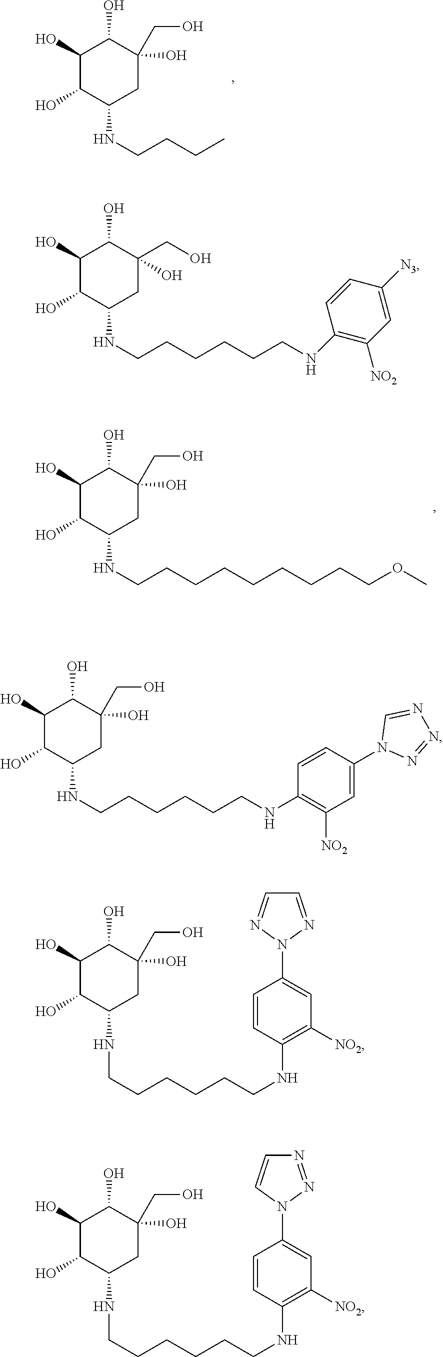 [SMILES: CCCCN[C@H]1C[C@](O)(CO)[C@@H](O)[C@H](O)[C@H]1O ; [N-]=[N+]=Nc1ccc(NCCCCCCN[C@H]2C[C@](O)(CO)[C@@H](O)[C@H](O)[C@H]2O)c([N+](=O)[O-])c1 ; COCCCCCCCCCN[C@H]1C[C@](O)(CO)[C@@H](O)[C@H](O)[C@H]1O ; O=[N+]([O-])c1cc(-n2cnnn2)ccc1NCCCCCCN[C@H]1C[C@](O)(CO)[C@@H](O)[C@H](O)[C@H]1O ; O=[N+]([O-])c1cc(-n2nccn2)ccc1NCCCCCCN[C@H]1C[C@](O)(CO)[C@@H](O)[C@H](O)[C@H]1O ; O=[N+]([O-])c1cc(-n2ccnn2)ccc1NCCCCCCN[C@H]1C[C@](O)(CO)[C@@H](O)[C@H](O)[C@H]1O]

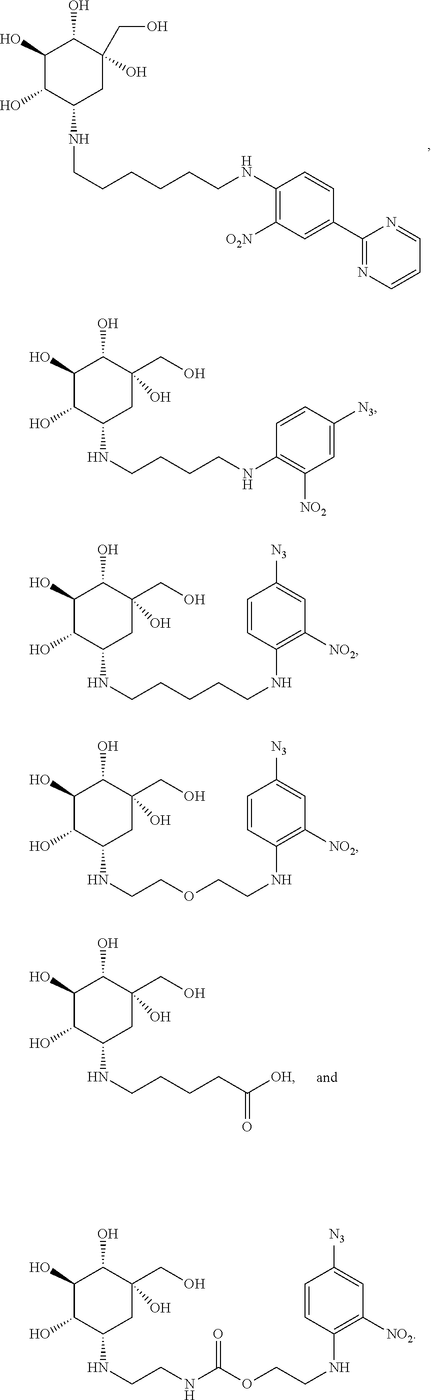 [SMILES: O=[N+]([O-])c1cc(-c2ncccn2)ccc1NCCCCCCN[C@H]1C[C@](O)(CO)[C@@H](O)[C@H](O)[C@H]1O ; [N-]=[N+]=Nc1ccc(NCCCCN[C@H]2C[C@](O)(CO)[C@@H](O)[C@H](O)[C@H]2O)c([N+](=O)[O-])c1 ; [N-]=[N+]=Nc1ccc(NCCCCCN[C@H]2C[C@](O)(CO)[C@@H](O)[C@H](O)[C@H]2O)c([N+](=O)[O-])c1 ; [N-]=[N+]=Nc1ccc(NCCOCCN[C@H]2C[C@](O)(CO)[C@@H](O)[C@H](O)[C@H]2O)c([N+](=O)[O-])c1 ; O=C(O)CCCCN[C@H]1C[C@](O)(CO)[C@@H](O)[C@H](O)[C@H]1O ; [N-]=[N+]=Nc1ccc(NCCOC(=O)NCCN[C@H]2C[C@](O)(CO)[C@@H](O)[C@H](O)[C@H]2O)c([N+](=O)[O-])c1]

In some embodiments, a pharmaceutical composition comprises the compound and at least one pharmaceutically acceptable excipient.

In some embodiments, a method of treating diabetes comprises administering to a subject in need thereof, a therapeutically effective amount of the compound.

In some embodiments, the subject is a mammal.

In some embodiments, the subject is a human.

In some embodiments, the diabetes is Type 1 diabetes.

In some embodiments, the diabetes is Type 2 diabetes.

In some embodiments, a method for inhibiting glycosidase function comprises administering to a subject in need thereof a therapeutically effective amount of the compound.

In some embodiments, a method for treating or preventing a viral infection comprises administering to a subject in need thereof a therapeutically effective amount of the compound.

In some embodiments, the viral infection is selected from the group consisting of hepatitis C virus (HCV) infection, hepatitis B virus (HBV) infection, dengue virus (DENV) infection, Marburg virus (MARV) infection, Ebola virus (EBOV) infection, BVHV , human immunodeficiency virus (HIV) infection, influenza A infection, influenza B infection, encephalitis viruses infection, Zika virus infection, and yellow fever virus (YFV) infection.

In some embodiments, the encephalitis virus infection is eastern equine encephalitis viruses virus infection, western equine encephalitis virus infection, and Japanese encephalitis virus (JEV) infection.

In some embodiments, the viral infection is dengue virus infection

In some embodiments, the viral infection is an influenza virus infection

In some embodiments, the influenza virus infection is an influenza A virus infection.

In some embodiments, the influenza virus infection is an influenza B virus infection.

### DETAILED DESCRIPTION

### Definitions

The indefinite articles “a,” “an,” and “the” include plural references unless clearly contradicted or the context clearly dictates otherwise.

As used herein, the term “alkylene” as used by itself or as part of another group refers to a straight-chain or branched-chain aliphatic bivalent hydrocarbon containing one to nine carbon atoms, i.e., a C₁-C₉ alkylene, or the number of carbon atoms designated, e.g., a C₁ alkylene such as methylene (—CH₂—), a C₂ alkylene such as ethylene (—CH₂—CH₂—), a C₃ alkylene such as propylene (—(CH₂)₃—) etc. Non-limiting exemplary C₁-C₉ alkylene groups include —CH₂—, —CH₂—CH₂—, —(CH₂)₃—, —(CH₂)₄—, —(CH₂)₅—, —(CH₂)₆—, —(CH₂)₇—, —(CH₂)₈—, and —(CH₂)₉—. In some embodiments, the alkylene can be a C₁-C₆ alkylene. In other embodiments, the alkylene can be a C₁-C₅ alkylene. In another embodiment, the alkylene can be a C₁-C₄ alkylene. In still further embodiments, the alkylene can be a C₁-C₃ alkylene, i.e., methylene (—CH₂—), ethylene (—CH₂—CH₂—), or propylene (—(CH₂)₃—).

As used herein, the term “optionally substituted alkylene” by itself or as part of another group refers to an alkylene group that is either unsubstituted or substituted with one, two, or more substituents selected from the group consisting of C₁-C₆ alkyl, C₁-C₆ alkoxy, —OH, —NH₂, halogen, —SH, and —NO₂—.

The term “alkyl” as used herein by itself or as part of another group refers to a straight-chain or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. Non-limiting exemplary C₁-C₆ alkyl groups include methyl, ethyl, propyl, isopropyl, butyl, se c-butyl, tent-butyl, isobutyl, 3-pentyl, and hexyl. In some embodiments, the alkyl can be a C₁-C₆ alkyl. In other embodiments, the alkyl can be a C₁-C₄ alkyl. In another embodiment, the alkyl can be a C₁-C₂ alkyl. In still further embodiments, the alkyl can be a C₁-C₃ alkyl, i.e., methyl, ethyl, propyl, or isopropyl.

As used herein, the term “alkoxy” by itself or as part of another group refers to an alkyl group attached to a terminal oxygen atom. Non-limiting exemplary alkoxy groups include methoxy, ethoxy, tert-butoxy, and the like. In some embodiments, the alkyl is a Cₓ-C_{y} alkyl, such as a C₁-C₆ alkyl and the resulting alkoxy is thus referred to as a “C₁-C₆ alkoxy.” Non-limiting exemplary alkoxy groups include methoxy, ethoxy, and tert-butoxy. In some embodiments, the alkyl can be a C₁-C₄ alkyl group, providing a C₁-C₄ alkoxy.

As used herein, the term “halogen” by itself or as part of another group refers to —Cl, —F, —Br, or —I.

As used herein, the term “heterocycle” refers to saturated and partially unsaturated, e.g., containing one or two double bonds, cyclic groups containing one, two, or three rings having from three to fourteen ring members, i.e., a 3- to 14-membered heterocycle, wherein at least one carbon atom of one of the rings is replaced with a heteroatom. Each heteroatom in the heterocycle is independently selected from the group consisting of oxygen, sulfur, and nitrogen atoms, wherein the sulfur and nitrogen atoms can optionally be oxidized (i.e. to a sulfoxide, sulfone, or N-oxide, as appropriate), or optionally quaternized in the case of nitrogen. The term “heterocycle” further includes groups wherein a ring —CH₂— is replaced with a —C(═O)—, such that the term “heterocycle” captures, for example, cyclic esters, cyclic ureido groups such as 2-imidazolidinone, and cyclic amide groups such as β-lactam, γ-lactam, δ-lactam, ε-lactam, and piperazin-2-one. In some embodiments, the heterocycle can be chosen from a 5- or 6-membered cyclic group containing one ring and one or two oxygen and/or nitrogen atoms. The heterocycle can be optionally linked to the rest of the molecule through any available carbon or nitrogen atom. Non-limiting exemplary heterocyclic groups include aziridinyl, azetidinyl, pyrrolidinyl, piperidinyl, piperazinyl, azepanyl, oxiranyl, oxetanyl, tetrahydrofuranyl, tetrahydrothienyl, tetrahydropyranyl, tetrahydrothiopyranyl, morpholinyl, thiomorpholinyl, thioxanyl, pyrrolinyl, 2H-pyranyl, 4H-pyranyl, dioxanyl, 1,3-dioxolanyl, pyrazolidinyl, pyrazolinyl, dihydropyranyl, dihydrothienyl, dihydrofuranyl, pyrazolidinyl, imidazolinyl, imidazolidinyl, dioxanyl, tetrahydropyranyl, 2-oxopyrrolidin-3-yl, piperazin-2-one, piperazine-2,6-dione, and 2-imidazolidinone.

As used herein, the term “heteroaryl” refers to an aromatic ring compound containing 5 or more ring members, of which, one or more is a heteroatom such as, but not limited to, N, O, and S. Non-limiting exemplary heteroaryl groups include pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, imidazolyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. In some embodiments, a heteroaryl group includes fused ring compounds and also includes heteroaryl groups that have other groups bonded to one of the ring members, such as alkyl groups, referred to as “substituted heteroaryl groups.” The heteroatom(s) may also be in oxidized form, if chemically possible. The point of attachment of a given heteroaryl group can be any appropriate atom. Exemplary heteroaryl groups showing various points of attachment suitable for use in the present compounds are shown below:

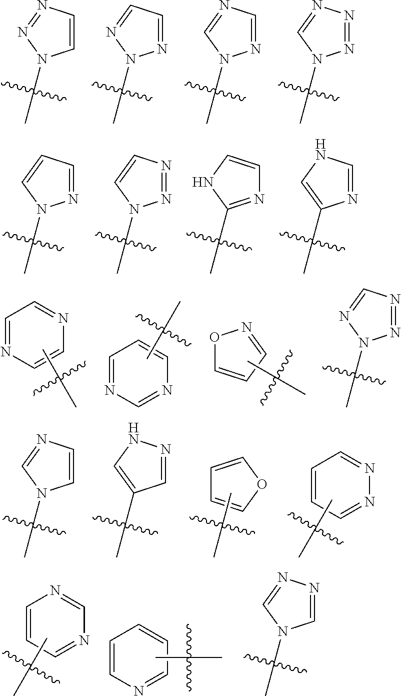

As used herein, the term “optional” or “optionally” means that the subsequently described circumstance may or may not occur, so that the description includes instances where the circumstance occurs and instances where it does not.

As used herein, the term “substituted” refers to independent replacement of one or more (typically 1, 2, 3, 4, or 5) hydrogen atoms on the substituted moiety with substituents independently selected from the group of substituents as specified for a particular group. In general, a non-hydrogen substituent can be any substituent that can be bound to an atom of the given moiety that is specified to be substituted.

As used herein, the phrase “pharmaceutically acceptable salt” refers to a salt formed with a compound described herein and a pharmaceutically acceptable acid. Pharmaceutically acceptable salts and methods for preparing salt forms are disclosed, for example, in Berge et al. (J. Pharm. Sci. 66:1-18, 1977). Examples of pharmaceutically acceptable acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts that can be formed with the compounds disclosed herein include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, glucoheptanoate, glycerolphsphate, hemisulfate, heptanoate, hexanoate, formate, succinate, fumarate, lactate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, thiocyanate, gluconate, methanesulfonate, ethanedisulfonate, benzene sulfonate, and p-toluenesulfonate salts.

### Description

Described herein are compounds that include a compound according to Formula (I):

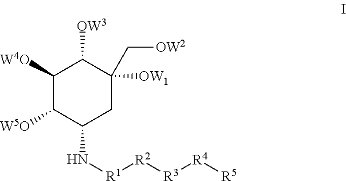

or a pharmaceutically acceptable salt thereof, wherein
W¹-W⁵ are each independently selected from the group consisting of —H, benzyl, —C(═O)—C₁-C₉ alkyl, and —C(═O)O—C₁-C₉ alkyl;
R¹ is optionally substituted C₁-C₉ alkylene;
R² is absent or selected from the group consisting of —H, —NH—, —O—, —C(═O)—, and —NH—C(═O)O —;
R³ is absent or selected from the group consisting of —O—, —C(═O)—, —C(═O)O —, and optionally substituted C₁-C₆ alkylene;
R⁴ is absent or selected from the group consisting of —H, —NH—, and optionally substituted C₁-C₆ alkylene; and
R⁵ is absent, or is

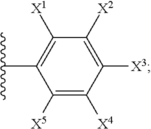

wherein X¹, X², X³, X⁴, and X⁵ are each independently selected from the group consisting of —H, —NO₂, —N₃, optionally subsittued C₂-C₁₂ heterocycle, and optionally substituted C₁-C₁₂ heteroaryl.

In some embodiments, W¹-W⁵ can each be independently selected from the group consisting of —H, —C(═O)—C₁-C₉ alkyl, and —C(═O)O—C₁-C₉ alkyl. In some embodiments, W¹-W⁵ can each be independently —C(═O)—C₁-C₉ alkyl. In some embodiments, W¹-W⁵ can each be independently —C(═O)O—C₁-C₉ alkyl. In some embodiments, W¹-W⁵ can each be independently —H.

In some embodiments, le can be optionally substituted C₁-C₉ alkylene. In some embodiments, le can be selected from the group consisting of —CH₂—, —CH₂—CH₂—, —(CH₂)₃—, —(CH₂)₄—, —(CH₂)₅—, —(CH₂)₆—, —(CH₂)₇—, —(CH₂)₈—, and —(CH₂)₉—. In certain embodiments, R¹ can be —CH₂—. In certain embodiments, R¹ can be —(CH₂)₂—. In certain embodiments, R¹ can be —(CH₂)₃—. In certain embodiments, R¹ can be —(CH₂)₄—. In certain embodiments, R¹ can be —(CH₂)₅—. In certain embodiments, R¹ can be —(CH₂)₆—. In certain embodiments, R¹ can be —(CH₂)₇—. In certain embodiments, R¹ can be —(CH₂)₈—. In certain embodiments, R¹ can be —(CH₂)₉—.

In some embodiments, R² can be absent or selected from the group consisting of —H, —NH—, —O—, —C(═O)—, and —NH—C(═O)O—. In certain embodiments, R² can be absent. In certain embodiments, R² can be —H. In certain embodiments, R² can be —NH—. In certain embodiments, R² can be —O—. In certain embodiments, R² can be —C(═O)—. In certain embodiments, R² can be —NH—C(═O)O—.

In some embodiments, R³ can be absent or selected from the group consisting of —O—, —C(═O)—, —C(═O)O—, and optionally substituted C₁-C₆ alkylene. In certain embodiments, R³ can be absent. In certain embodiments, R³ can be —O—. In certain embodiments, R³ can be —C(═O)—. In certain embodiments, R³ can be —C(═O)O—.

In certain embodiments, R³ can be a C₁-C₆ alkylene. In certain embodiments, R³ can be selected from the group consisting of —CH₂—, —CH₂—CH₂—, —(CH₂)₃—, —(CH₂)₄—, —(CH₂)₅—, and —(CH₂)₆—. In certain embodiments, R³ can be —CH₂—. In certain embodiments, R³ can be —CH₂—CH₂—. In certain embodiments, R³ can be —(CH₂)₃—. In certain embodiments, R³ can be —(CH₂)₄—. In certain embodiments, R³ can be —(CH₂)₅—. In certain embodiments, R³ can be —(CH₂)₆—.

In some embodiments, R⁴ can be absent or selected from the group consisting of —H, —NH—, and optionally substituted C₁-C₆ alkylene. In certain embodiments, R⁴ can be absent. In certain embodiments, R⁴ can be —H. In certain embodiments, R⁴ can be —NH—.

In certain embodiments, R⁴ can be a C₁-C₆ alkylene. In some embodiments, the R⁴ can be selected from the group consisting of —CH₂—, —CH₂—CH₂—, —(CH₂)₃—, —(CH₂)₄—, —(CH₂)₅—, and —(CH₂)₆—. In certain embodiments, R⁴ can be —CH₂—. In certain embodiments, R⁴ can be —CH₂—CH₂—. In certain embodiments, R⁴ can be —(CH₂)₃—. In certain embodiments, R⁴ can be —(CH₂)₄—. In certain embodiments, R⁴ can be —(CH₂)₅—. In certain embodiments, R⁴ can be —(CH₂)₆—.

In some embodiments, R⁵ can be absent, or can be

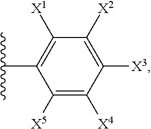

wherein X¹, X², X³, X⁴, and X⁵ can each be independently selected from the group consisting of —H, —NO₂, —N₃, optionally substituted C₂-C₁₂ heterocycle, and optionally substituted C₁-C₁₂ heteroaryl.

In certain embodiments, R⁵ can be absent. In certain embodiments, R⁵ can be

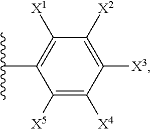

wherein X¹, X², X³, X⁴, and X⁵ can each be independently selected from the group consisting of —H, —NO₂, —N₃, optionally substituted C₂-C₁₂ heterocycle, and optionally substituted C₁-C₁₂ heteroaryl. In certain embodiments, the optionally substituted C₁-C₁₂ heteroaryl can be selected from the group consisting of

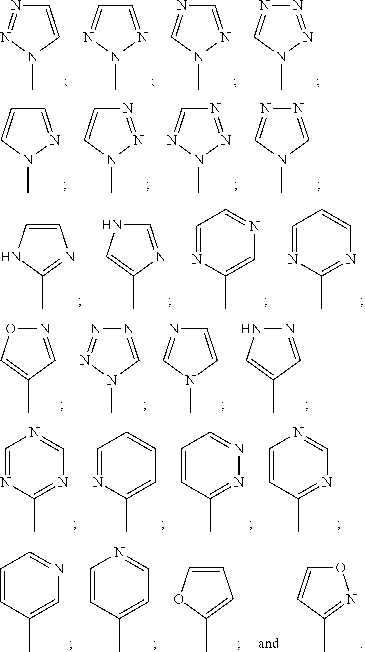

In certain embodiments, the optionally substituted C₂-C₁₂ heterocycle can be selected from the group consisting of

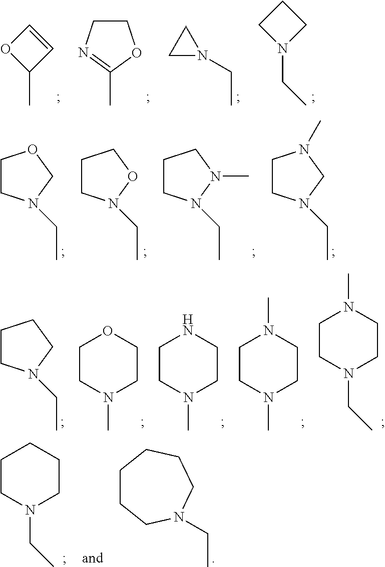

In certain embodiments, the optionally substituted C₁-C₁₂ heteroaryl can be selected from the group consisting of

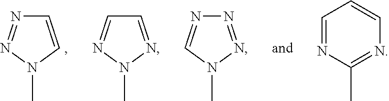

In certain embodiments, the C₂-C₁₂ heterocycle can be selected from the group consisting of

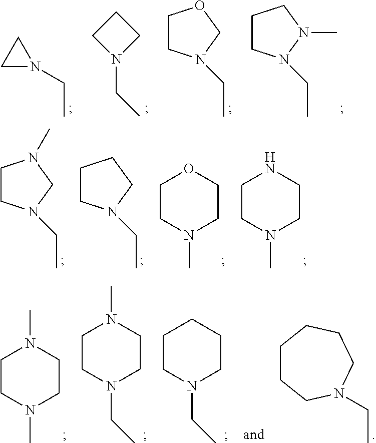

In certain embodiments, R⁵ can be

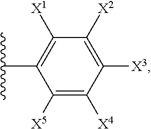

wherein X¹ can be —NO₂. In certain embodiments, R⁵ can be

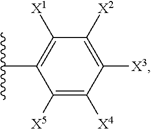

wherein X², X⁴, and X⁵ can each be independently —H. In certain embodiments, R⁵ can be

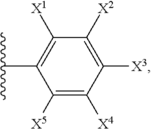

wherein X¹ can be —NO₂, X², X⁴, and X⁵ can each be independently —H, and X³ can be —N₃.

In certain embodiments, R⁵ can be

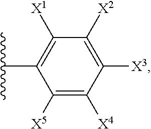

wherein X¹ can be —NO₂, X², X⁴, and X⁵ can each be independently —H, and X³ can be

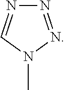

In certain embodiments, R⁵ can be

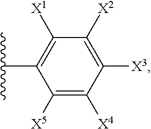

wherein X¹ can be —NO₂, X², X⁴, and X⁵ can each be independently —H, and X³ can be

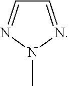

In certain embodiments, R⁵ can be

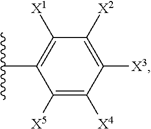

wherein X¹ can be —NO₂, X², X⁴, and X⁵ can each be independently —H, and X³ can be

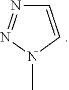

In certain embodiments, R⁵ can be

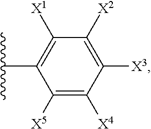

wherein X¹ can be —NO₂, X², X⁴, and X⁵ can each be independently —H, and X³ can be

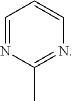

Embodiments according to Formula (I) are illustrated in Table 1. Table 1 is provided for exemplary purposes, and is not to be considered as an all-inclusive list of compounds. Additionally, while Table 1 describes the free-base embodiments of compounds 1-12, the present disclosure includes any and all appropriate pharmaceutically acceptable salts of these compounds.

TABLE 1

| Cmpd | | |
|---|---|---|
| # | Structure | Name |
| 1 | 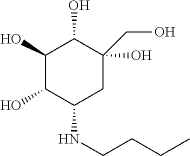 [SMILES: CCCCN[C@H]1C[C@](O)(CO)[C@@H](O)[C@H](O)[C@H]1O] | (1S,2S,3R,4S,5S)-5-(butylamino)-1- (hydroxymethyl)cyclohexane-1,2,3,4- tetraol |
| 2 | 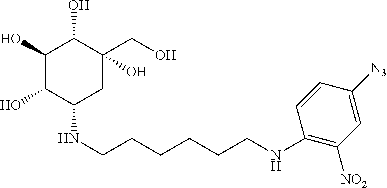 [SMILES: [N-]=[N+]=Nc1ccc(NCCCCCCN[C@H]2C[C@](O)(CO)[C@@H](O)[C@H](O)[C@H]2O)c([N+](=O)[O-])c1] | (1S,2S,3R,4S,5S)-5-((6-((4-azido-2- nitrophenyl)amino)hexyl)amino)-1- (hydroxymethyl)cyclohexane-1,2,3,4- tetraol |
| 3 | 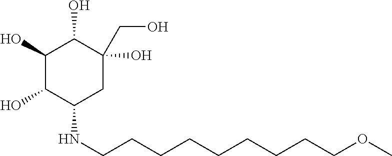 [SMILES: COCCCCCCCCCN[C@H]1C[C@](O)(CO)[C@@H](O)[C@H](O)[C@H]1O] | (1S,2S,3R,4S,5S)-1-(hydroxymethyl)-5- ((9-methoxynonyl)amino)cyclohexane- 1,2,3,4-tetraol |
| 4 | 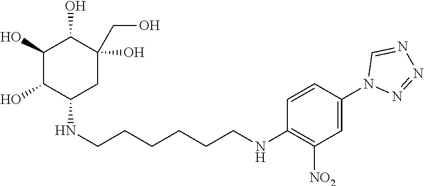 [SMILES: O=[N+]([O-])c1cc(-n2cnnn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(1H-tetrazol-1- yl)phenyl)amino)hexyl)amino)cyclohexane- 1,2,3,4-tetraol |
| 5 | 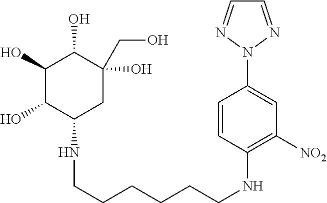 [SMILES: O=[N+]([O-])c1cc(-n2nccn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(2H-1,2,3-triazol-2- yl)phenyl)amino)hexyl)amino)cyclohexane- 1,2,3,4-tetraol |
| 6 | 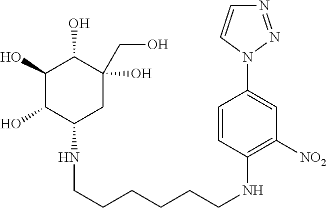 [SMILES: O=[N+]([O-])c1cc(-n2ccnn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(1H-1,2,3-triazol-1- yl)phenyl)amino)hexyl)amino)cyclohexane- 1,2,3,4-tetraol |
| 7 | 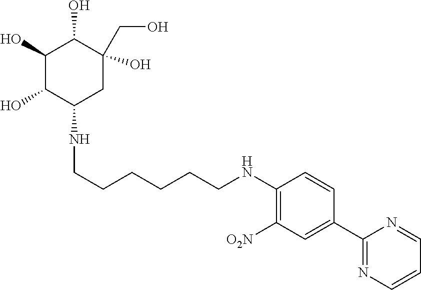 [SMILES: O=[N+]([O-])c1cc(-c2ncccn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(pyrimidin-2- yl)phenyl)amino)hexyl)amino)cyclohexane- 1,2,3,4-tetraol |
| 8 | 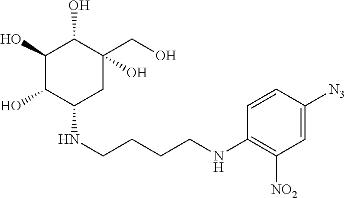 [SMILES: [N-]=[N+]=Nc1ccc(NCCCCN[C@H]2C[C@](O)(CO)[C@@H](O)[C@H](O)[C@H]2O)c([N+](=O)[O-])c1] | (1S,2S,3R,4S,5S)-5-((4-((4-azido-2- nitrophenyl)amino)butyl)amino)-1- (hydroxymethyl)cyclohexane-1,2,3,4- tetraol |
| 9 | 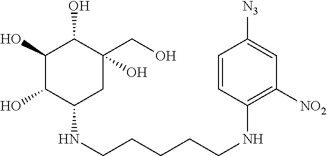 [SMILES: [N-]=[N+]=Nc1ccc(NCCCCCN[C@H]2C[C@](O)(CO)[C@@H](O)[C@H](O)[C@H]2O)c([N+](=O)[O-])c1] | (1S,2S,3R,4S,5S)-5-((5-((4-azido-2- nitrophenyl)amino)pentyl)amino)-1- (hydroxymethyl)cyclohexane-1,2,3,4- tetraol |
| 10 | 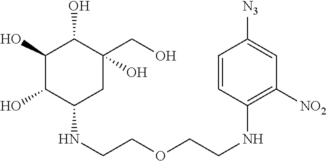 [SMILES: [N-]=[N+]=Nc1ccc(NCCOCCN[C@H]2C[C@](O)(CO)[C@@H](O)[C@H](O)[C@H]2O)c([N+](=O)[O-])c1] | (1S,2S,3R,4S,5S)-5-((2-(2-((4-azido-2- nitrophenyl)amino)ethoxy)ethyl)amino)- 1-(hydroxymethyl)cyclohexane-1,2,3,4- tetraol |
| 11 | 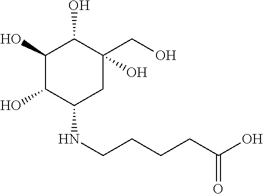 [SMILES: O=C(O)CCCCN[C@H]1C[C@](O)(CO)[C@@H](O)[C@H](O)[C@H]1O] | 5-(((1S,2S,3R,4S,5S)-2,3,4,5- tetrahydroxy-5- (hydroxymethyl)cyclohexyl)amino) pentanoic acid |
| 12 | 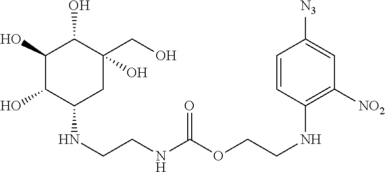 [SMILES: [N-]=[N+]=Nc1ccc(NCCOC(=O)NCCN[C@H]2C[C@](O)(CO)[C@@H](O)[C@H](O)[C@H]2O)c([N+](=O)[O-])c1] | 2-((4-azido-2-nitrophenyl)amino)ethyl (2-(((1S,2S,3R,4S,5S)-2,3,4,5- tetrahydroxy-5- (hydroxymethyl)cyclohexyl)amino)ethyl) carbamate |

In some embodiments, the compound of Formula (I) can be a compound of Formula (II):

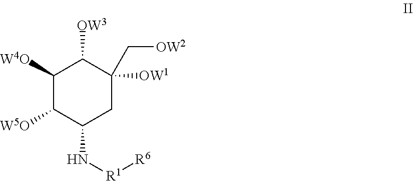

or a pharmaceutically acceptable salt thereof, wherein
W¹-W⁵ are each independently selected from the group consisting of —H, —C(═O)—C₁-C₉ alkyl, and —C(═O)O—C₁-C₉ alkyl;
R¹ is optionally substituted C₁-C₉ alkylene;
R⁶ is selected from the group consisting of —H, —OR⁷, and —C(═O)—OR⁸; wherein each of R⁷ and R⁸ is —H or a C₁-C₆ alkyl.

In some embodiments, W¹-W⁵ can each be independently selected from the group consisting of —H, —C(═O)—C₁-C₉ alkyl, and —C(═O)O—C₁-C₉ alkyl. In some embodiments, W¹-W⁵ can each be independently —C(═O)—C₁-C₉ alkyl. In some embodiments, W¹-W⁵ can each be independently —C(═O)O—C₁-C₉ alkyl. In some embodiments, W¹-W⁵ can each be independently —H.

In some embodiments, R¹ can be optionally substituted C₁-C₉ alkylene. In some embodiments, R¹ can be selected from the group consisting of —CH₂—, —CH₂—CH₂—, —(CH₂)₃—, —(CH₂)₄—, —(CH₂)₅—, —(CH₂)₆—, —(CH₂)₇—, —(CH₂)₈—, and —(CH₂)₉—. In certain embodiments, R¹ can be —CH₂—. In certain embodiments, le can be —(CH₂)₂—. In certain embodiments, R¹ can be —(CH₂)₃—. In certain embodiments, R¹ can be —(CH₂)₄—. In certain embodiments, R¹ can be —(CH₂)₅—. In certain embodiments, R¹ can be —(CH₂)₆—. In certain embodiments, R¹ can be —(CH₂)₇—. In certain embodiments, R¹ can be —(CH₂)₈—. In certain embodiments, R¹ can be —(CH₂)₉—.

In some embodiments, R⁶ can be —H.

In some embodiments, R⁶ can be —OR⁷. In some embodiments, R⁷ can —H or a C₁-C₆ alkyl. In some embodiments, R⁷ can —H. In some embodiments, R⁷ can be a C₁-C₆ alkyl such as methyl, ethyl, n-propyl, isopropyl, butyl, sec-butyl, t-butyl, n-pentyl, or n-hexyl. In some embodiments, the C₁-C₆ alkyl can be linear, branched, or cyclic. In certain embodiments, the C₁-C₆ alkyl is linear. In particular embodiments, the C₁-C₆ alkyl can be methyl.

In some embodiments, R⁶ can be —C(═O)—OR⁸. In some embodiments, R⁸ can —H or a C₁-C₆ alkyl. In some embodiments, R⁸ can be a C₁-C₆ alkyl such as methyl, ethyl, n-propyl, isopropyl, butyl, sec-butyl, t-butyl, n-pentyl, or n-hexyl. In some embodiments, the C₁-C₆ alkyl can be linear, branched, or cyclic. In certain embodiments, the C₁-C₆ alkyl is linear. In particular embodiments, R⁸ can —H.

Embodiments according to Formula (II) are illustrated in Table 2. Table 2 is provided for exemplary purposes, and is not to be considered as an all-inclusive list of compounds. Additionally, while Table 2 describes the free-base embodiments of compounds 1, 3, and 11, the present disclosure includes any and all appropriate pharmaceutically acceptable salts of these compounds.

TABLE 2

| Cmpd | | |
|---|---|---|
| # | Structure | Name |
| 1 | 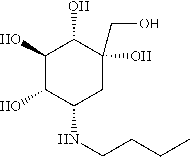 [SMILES: CCCCN[C@H]1C[C@](O)(CO)[C@@H](O)[C@H](O)[C@H]1O] | (1S,2S,3R,4S,5S)-5-(butylamino)-1- (hydroxymethyl)cyclohexane-1,2,3,4- tetraol |
| 3 | 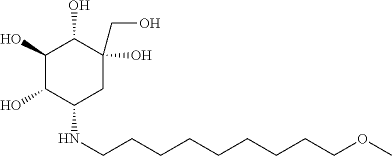 [SMILES: COCCCCCCCCCN[C@H]1C[C@](O)(CO)[C@@H](O)[C@H](O)[C@H]1O] | (1S,2S,3R,4S,5S)-1-(hydroxymethyl)-5- ((9-methoxynonyl)amino)cyclohexane- 1,2,3,4-tetraol |
| 11 | 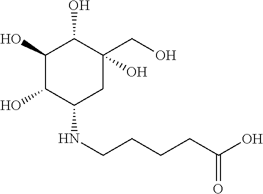 [SMILES: O=C(O)CCCCN[C@H]1C[C@](O)(CO)[C@@H](O)[C@H](O)[C@H]1O] | 5-(((1S,2S,3R,4S,5S)-2,3,4,5- tetrahydroxy-5- (hydroxymethyl)cyclohexyl)amino) pentanoic acid |

In some embodiments, the compound of Formula (I) is not a compound of Formula (II):

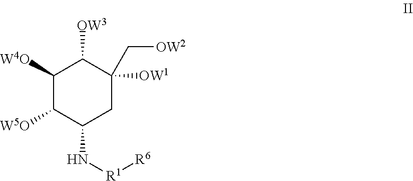

or a pharmaceutically acceptable salt thereof, wherein W₁-W₅, R⁶ and R⁶ have the meanings described herein. In some embodiments, the compound of Formula I is not a compound according to Table 2, or a pharmaceutically acceptable salt thereof.

In some embodiments, the compound of Formula (I) can be a compound of Formula (III):

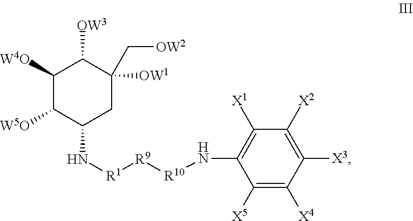

or a pharmaceutically acceptable salt thereof, wherein
W¹-W⁵ are each independently selected from the group consisting of —H, —C(═O)—C1-C9 alkyl, and —C(═O)O—C1-C9 alkyl;
R1 is optionally substituted C1-C9 alkylene;
R9 is absent or selected from the group consisting of —NH—, —O—, —C(═O)—, —C(═O)O—, —NH—C(═O)—, and —NH—C(═O)O—;
R10 is absent or optionally substituted C1-C6 alkylene;
wherein X1,X2, X3, X4, and X5 are each independently selected from the group consisting of —H, —NO₂, —N₃, optionally substituted C₂-C₁₂ heterocycle, and optionally substituted C₁-C₁₂ heteroaryl.

In some embodiments, W¹-W⁵ can each be independently selected from the group consisting of —H, —C(═O)—C₁-C₉ alkyl, and —C(═O)O—C₁-C₉ alkyl. In some embodiments, W¹-W⁵ can each be independently —C(═O)—C₁-C₉ alkyl. In some embodiments, W¹-W⁵ can each be independently —C(═O)O—C₁-C₉ alkyl. In some embodiments, W¹-W⁵ can each be independently —H.

In some embodiments, le can be optionally substituted C₁-C₉ alkylene. In some embodiments, R¹ can be selected from the group consisting of —CH₂—, —CH₂—CH₂—, —(CH₂)₃—, —(CH₂)₄—, —(CH₂)₅—, —(CH₂)₆—, —(CH₂)₇—, —(CH₂)₈—, and —(CH₂)₉—. In certain embodiments, R¹ can be -—CH₂—. In certain embodiments, R¹ can be —(CH₂)₂—. In certain embodiments, R¹ can be -—(CH₂)₃—. In certain embodiments, R¹ can be —(CH₂)₄—. In certain embodiments, R¹ can be —(CH₂)₅—. In certain embodiments, R¹ can be —(CH₂)₆—. In certain embodiments, R¹ can be —(CH₂)₇—. In certain embodiments, R¹ can be —(CH₂)₈—. In certain embodiments, R¹ can be —(CH₂)₉—.

In some embodiments, R⁹ can be absent. In some embodiments, R⁹ can be —NH—. In some embodiments, R⁹ can be —NH—. In some embodiments, R⁹ can be —O—. In some embodiments, R⁹ can be —C(═O)—. In some embodiments, R⁹ can be —C(═O)O—. In some embodiments, R⁹ can be —NH—C(═O)—. In some embodiments, R⁹ can be —NH—C(═O)O—.

In some embodiments, R¹⁰ can be absent. In some embodiments, R¹⁰ can be optionally substituted C₁-C₆ alkylene; In some embodiments, R¹⁰ can be selected from the group consisting of —CH₂—, —CH₂—CH₂—, —(CH₂)₃—, —(CH₂)₄—, —(CH₂)₅—, and —(CH₂)₆—. In certain embodiments, R¹⁰ can be —CH₂—. In certain embodiments, R¹⁰ can be —(CH₂)₂—. In certain embodiments, R¹⁰ can be —(CH₂)₃—. In certain embodiments, R¹⁰ can be —(CH₂)₄—. In certain embodiments, R¹⁰ can be —(CH₂)₅—. In certain embodiments, R¹⁰ can be —(CH₂)₆—.

In certain embodiments, X¹, X², X³, X⁴, and X⁵ can each be independently selected from the group consisting of —H, —NO₂, —N₃, optionally substituted C₂C₁₂ heterocycle, and optionally substituted C₁C₁₂ heteroaryl. In certain embodiments, the optionally substituted C₁C₁₂ heteroaryl can be selected from the group consisting of

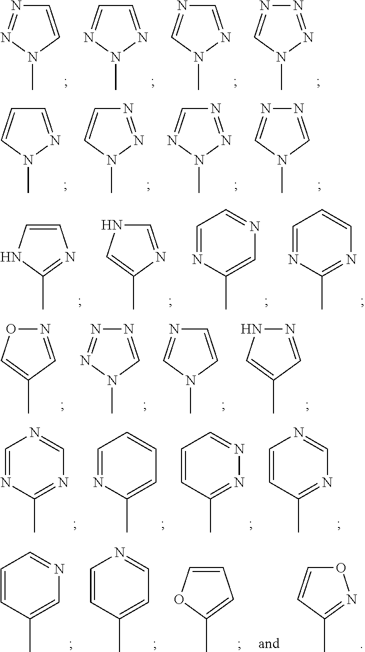

In certain embodiments, the optionally substituted C₂C₁₂ heterocycle can be selected from the group consisting of

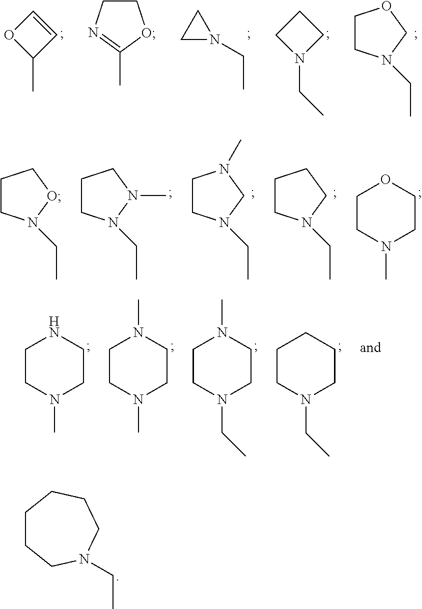

In certain embodiments, the optionally substituted C₁C₁₂ heteroaryl can be selected from the group consisting of

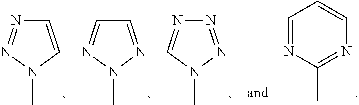

In certain embodiments, the optionally substituted C₂C₁₂ heterocycle can be selected from the group consisting of

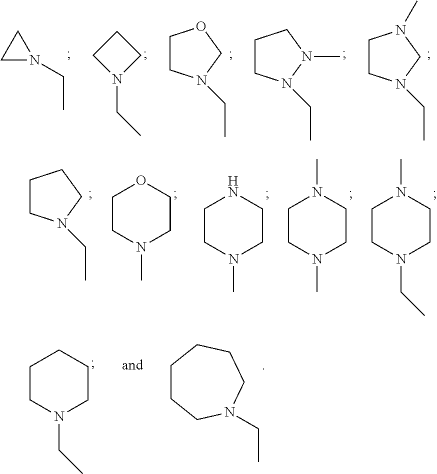

In certain embodiments, X¹ can be —NO₂ In certain embodiments, X², X⁴, and X⁵ can each be independently —H. In certain embodiments, X¹ can be —NO₂, X², X⁴, and X⁵ can each be independently —H, and X³ can be —N₃.

In certain embodiments, X¹ can be —NO₂, X², X⁴, and X⁵ can each be independently —H, and X³ can be

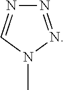

In certain embodiments, X¹ can be —NO₂, X², X⁴, and X⁵ can each be independently —H, and X³ can be

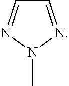

In certain embodiments, X¹ can be —NO₂, X², X⁴, and X⁵ can each be independently —H, and X³ can be

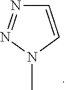

In certain embodiments, X¹ can be —NO₂, X², X⁴, and X⁵ can each be independently —H, and X³ can be

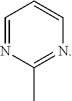

Exemplary compounds according to Formula (III) are illustrated in Table 3. Table 3 is provided for exemplary purposes, and is not to be considered as an all-inclusive list of compounds. Additionally, while Table 3 describes the free-base embodiments of compounds 2, 4, 5,6, 7, 8, 9, 10, and 12, the present disclosure includes any and all appropriate pharmaceutically acceptable salts of these compounds.

TABLE 3

| Cmpd | | |
|---|---|---|
| # | Structure | Name |
| 2 | 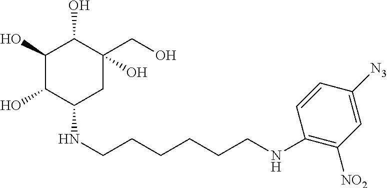 [SMILES: [N-]=[N+]=Nc1ccc(NCCCCCCN[C@H]2C[C@](O)(CO)[C@@H](O)[C@H](O)[C@H]2O)c([N+](=O)[O-])c1] | (1S,2S,3R,4S,5S)-5-((6-((4-azido-2- nitrophenyl)amino)hexyl)amino)-1- (hydroxymethyl)cyclohexane-1,2,3,4- tetraol |
| 4 | 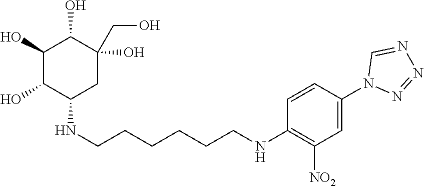 [SMILES: O=[N+]([O-])c1cc(-n2cnnn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(1H-tetrazol-1- yl)phenyl)amino)hexyl)amino) cyclohexane-1,2,3,4-tetraol |
| 5 | 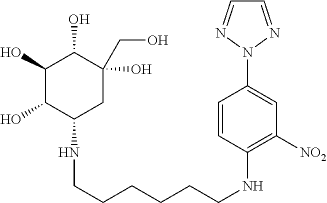 [SMILES: O=[N+]([O-])c1cc(-n2nccn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(2H-1,2,3-triazol-2- yl)phenyl)amino)hexyl)amino) cyclohexane-1,2,3,4-tetraol |
| 6 | 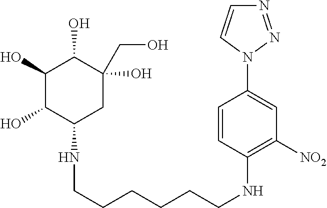 [SMILES: O=[N+]([O-])c1cc(-n2ccnn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(1H-1,2,3-triazol-1- yl)phenyl)amino)hexyl)amino) cyclohexane-1,2,3,4-tetraol |
| 7 | 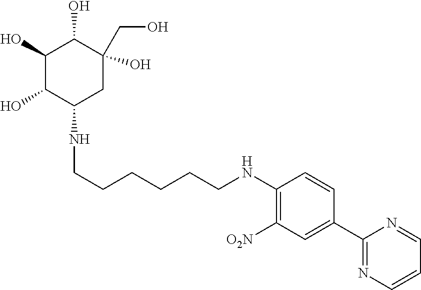 [SMILES: O=[N+]([O-])c1cc(-c2ncccn2)ccc1NCCCCCCN[C@H]1C[C@](O)(CO)[C@@H](O)[C@H](O)[C@H]1O] | (1S,2S,3R,4S,5S)-1-(hydroxymethyl)-5- ((6-((2-nitro-4-(pyrimidin-2- yl)phenyl)amino)hexyl)amino) cyclohexane-1,2,3,4-tetraol |
| 8 | 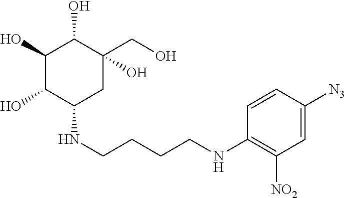 [SMILES: [N-]=[N+]=Nc1ccc(NCCCCN[C@H]2C[C@](O)(CO)[C@@H](O)[C@H](O)[C@H]2O)c([N+](=O)[O-])c1] | (1S,2S,3R,4S,5S)-5-((4-((4-azido-2- nitrophenyl)amino)butyl)amino)-1- (hydroxymethyl)cyclohexane-1,2,3,4- tetraol |
| 9 | 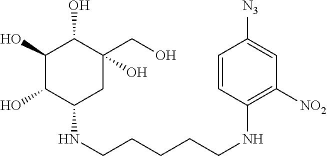 [SMILES: [N-]=[N+]=Nc1ccc(NCCCCCN[C@H]2C[C@](O)(CO)[C@@H](O)[C@H](O)[C@H]2O)c([N+](=O)[O-])c1] | (1S,2S,3R,4S,5S)-5-((5-((4-azido-2- nitrophenyl)amino)pentyl)amino)-1- (hydroxymethyl)cyclohexane-1,2,3,4- tetraol |
| 10 | 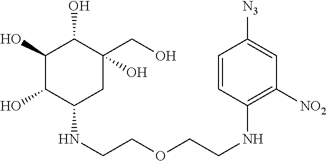 [SMILES: [N-]=[N+]=Nc1ccc(NCCOCCN[C@H]2C[C@](O)(CO)[C@@H](O)[C@H](O)[C@H]2O)c([N+](=O)[O-])c1] | (1S,2S,3R,4S,5S)-5-((2-(2-((4-azido-2- nitrophenyl)amino)ethoxy)ethyl)amino)- 1-(hydroxymethyl)cyclohexane-1,2,3,4- tetraol |
| 12 | 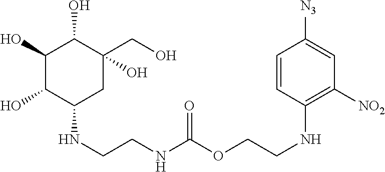 [SMILES: [N-]=[N+]=Nc1ccc(NCCOC(=O)NCCN[C@H]2C[C@](O)(CO)[C@@H](O)[C@H](O)[C@H]2O)c([N+](=O)[O-])c1] | 2-((4-azido-2-nitrophenyl)amino)ethyl (2-(((1S,2S,3R,4S,5S)-2,3,4,5- tetrahydroxy-5- (hydroxymethyl)cyclohexyl)amino)ethyl) carbamate |

In some embodiments, the compound of Formula (III) can be a compound of Formula (III-A), (III-B), or (III-C):

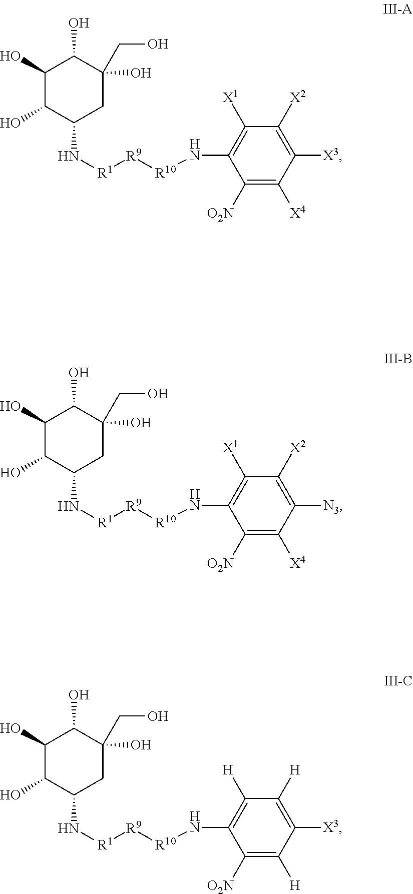

wherein R¹, R⁹, R¹⁰, X¹, X², X³, and X⁴ have the meanings described herein. In particular embodiments, X³ can be optionally substituted C₁C₁₂ heteroaryl. In certain embodiments, In certain embodiments, the optionally substituted C₁C₁₂ heteroaryl can be selected from the group consisting of

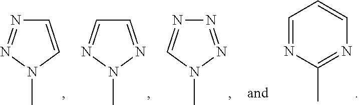

In some embodiments, a compound of the present disclosure can be a part of a pharmaceutical composition, which can also include one or more pharmaceutically acceptable excipients. Suitable excipients are discussed, for example, in Gennaro, A. R., Remington's Pharmaceutical Sciences, Mack Publishing Co., Easton, P.A. (18th ed, 1995), and Liberman, Hours. A. and Lachman, L, Eds., Pharmaceutical Dosage Forms, Marcel Dekker Inc., New York, N.Y. (1980).

Exemplary pharmaceutically acceptable excipients include, but are not limited to diluents, binders, fillers, buffering agents, pH modifying agents, disintegrants, dispersants, preservatives, lubricants, taste-masking agents, flavoring agents, coloring agents. The amounts and types of excipients utilized to form pharmaceutical compositions can be selected according to known principles of pharmaceutical science, as well as the desired route of administration, i.e. orally, parenterally (i.e. via subcutaneous, intravenous, intramuscular, intra-articular, intrasternal injection, or other infusion technique), bucally, sublingually, etc.

Exemplary diluents include, but are not limited to those diluents that are compressible (i.e., plastically deformable) and those that are abrasively brittle. Exemplary compressible diluents include, but are not limited to, microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, trehalose, and combinations thereof. Abrasively brittle diluents include, but are not limited to, dibasic calcium phosphate (anhydrous or dihydrate), calcium phosphate tribasic, calcium carbonate, magnesium carbonate, and combinations thereof.

Exemplary binders include, but are not limited to, starches (for example, maize starch, wheat starch, rice starch, or potato starch), pregelatinized starches, gelatin, polyvinylpyrrolidone, tragacanth, polyvinyl pyrrolidone, cellulose, methylcellulose, hydroxypropylmethylcellulose, sodium carboxymethylcellulose, ethylcellulose, polyacrylamides, polyvinyloxoazolidone, polyvinylalcohols, C₁₂C₁₈ fatty acid alcohol, polyethylene glycol, polyols, saccharides, oligosaccharides, polypeptides, oligopeptides, and combinations thereof.

Suitable fillers include, but are not limited to, saccharides (for example, lactose, sucrose, mannitol or sorbitol), cellulose preparations, calcium phosphates (for example, tricalcium phosphate or calcium hydrogen phosphate), and combinations thereof.

Suitable buffering agents include, but are not limited to, phosphates, carbonates, citrates, tris buffers, and buffered saline salts (e.g., tris buffered saline or phosphate buffered saline) and combinations thereof, as appropriate.

Suitable pH modifiers include, but are not limited to, sodium carbonate, sodium bicarbonate, sodium citrate, citric acid, phosphoric acid, and combinations thereof.

Suitable disintegrants include, but are not limited to, non-effervescent and effervescent disintegrants. Exemplary non-effervescent disintegrants include, but are not limited to, starches such as corn starch, potato starch, pregelatinized and modified starches; sweeteners; clays such as bentonite; micro-crystalline cellulose; alginates; sodium starch glycolate; and gums such as agar, guar, locust bean, karaya, pectin, and tragacanth. Exemplary effervescent disintegrants include, but are not limited to, sodium bicarbonate in combination with citric acid, and sodium bicarbonate in combination with tartaric acid. Combinations of any of the foregoing can also be used.

Exemplary dispersants include, but are not limited to, starch, alginic acid, polyvinylpyrrolidones, guar gum, kaolin, bentonite, purified wood cellulose, sodium starch glycolate, isoamorphous silicate, microcrystalline cellulose, and combinations thereof.

Exemplary preservatives include, but are not limited to, antioxidants, such as BHA, BHT, vitamin A, vitamin C, vitamin E, retinyl palmitate, citric acid, and sodium citrate; chelators, such as EDTA, and EGTA; and antimicrobials, such as parabens, chlorobutanol, and phenol. Combinations of any of the foregoing can also be used.

Exemplary lubricants include, but are not limited to, minerals such as talc and silica; fats such as vegetable stearin, magnesium stearate, and stearic acid. Combinations of any of the foregoing can also be used.

Exemplary taste-masking agents include, but are not limited to, cellulose ethers, polyethylene glycols, polyvinyl alcohol, polyvinyl alcohol and polyethylene glycol copolymers, monoglycerides, triglycerides, acrylic polymers, mixtures of acrylic polymers and cellulose ethers, cellulose acetate phthalate, and combinations thereof.

Exemplary flavoring agents include, but are not limited to, synthetic flavor oils, flavoring aromatics, natural oils, and extracts from plants, leaves, flowers, fruits, and combinations thereof.

Exemplary coloring agents include, but are not limited to, food, drug and cosmetic colors (FD&C), drug and cosmetic colors (D&C), and external drug and cosmetic colors (Ext. D&C).

In some embodiments, the composition can be formulated into various dosage forms and administered by a number of different means that will deliver a therapeutically effective amount of the active ingredient.

In some embodiments, topical administration can involve the use of transdermal administration such as transdermal patches, or iontophoresis devices.

In some embodiments, the pharmaceutical composition can be a solid dosage forms for oral administration such as a capsule, tablet, caplet, pill, powder, pellet, or granule. In such solid dosage forms, the active ingredient can be ordinarily combined with one or more pharmaceutically acceptable excipients, examples of which are detailed above. Oral preparations can also be administered as aqueous suspensions, elixirs, or syrups. The active ingredient can be combined with various sweetening or flavoring agents, coloring agents, emulsifying and/or suspending agents, and diluents such as water, ethanol, glycerin, and combinations thereof. For administration by inhalation, the compounds can be delivered in the form of an aerosol spray from pressured container, or dispenser which contains a suitable propellant, e.g., a gas such as carbon dioxide, or a nebulizer.

In some embodiments, the preparation can be an aqueous or an oil-based solution for parenteral administration. The parenteral administration can be subcutaneous, intradermal, intravenous, intramuscular, intra-articular, or intraperitoneal. Aqueous solutions can include a sterile diluent such as water, saline solution, a pharmaceutically acceptable polyol such as glycerol and propylene glycol, and other synthetic solvents; an antibacterial and/or antifungal agent such as benzyl alcohol, methyl paraben, chlorobutanol, phenol, thimerosal, and the like; an antioxidant such as ascorbic acid and sodium bisulfate; a chelating agent such as ethylenediaminetetraacetic acid; a buffer such as acetate, citrate, and phosphate; and/or an agent for the adjustment of tonicity such as sodium chloride, dextrose, and a polyalcohol such as mannitol and sorbitol. The pH of the aqueous solution can be adjusted with acids or bases such as hydrochloric acid or sodium hydroxide. Oil-based solutions or suspensions can further comprise sesame, peanut, olive oil, and mineral oil. The compositions can be presented in unit-dose or multi-dose containers, such as sealed ampoules and vials, and can be stored in a freeze-dried (lyophilized) condition, which requires the addition of the sterile liquid carried, such as water for injections, immediately prior to use. Extemporaneous injection solutions and suspensions can be prepared from sterile powders, granules, or tablets.

In some embodiments, penetrants appropriate to the barrier to be permeated can be generally included in the preparation for topical (e.g., transdermal or transmucosal) administration. Pharmaceutical compositions adapted for topical administration can be formulated as ointments, creams, suspensions, lotions, powders, solutions, pastes, gels, sprays, aerosols, or oils. In some embodiments, the pharmaceutical composition can be applied as a topical ointment or cream. When formulated in an ointment, the active ingredient can be employed with either a paraffinic or a water-miscible ointment base. Alternatively, the active ingredient can be formulated in a cream with an oil-in-water cream base or a water-in-oil base. Pharmaceutical compositions adapted for topical administration to the eye include eye drops, wherein the active ingredient can be dissolved or suspended in a suitable carrier, especially an aqueous solvent. Pharmaceutical compositions adapted for topical administration in the mouth include lozenges, pastilles, and mouth washes. Transmucosal administration can be accomplished through the use of nasal sprays, aerosol sprays, tablets, or suppositories. Transdermal administration can be accomplished through the use of ointments, salves, gels, patches, or creams.

In some embodiments, subjects can be, but is not limited to, a human, and/or a companion animal such as a cat, dog, rodent, or horse; a research animal such as a rabbit, sheep, pig, dog, non-human primate (such as chimpanzees, monkeys, and gorillas), mouse, rat or other rodent; an agricultural animal such as a cow, pig, goat, deer, chicken or other fowl; and a zoo animal; The subject can be of any age without limitation. In some embodiments, the subject can be a human.

In some embodiments, a compound of the present disclosure can be administered in a therapeutically effective amount, either on its own, or formulated as a composition, which includes prophylactic amounts or lower dosages, for example, when combined with another agent. As used herein, “an effective amount” refers to doses of compound sufficient to provide circulating concentrations high enough to impart a beneficial effect on the recipient thereof. The precise amount to be administered can be determined by the skilled practitioner in view of desired dosages, side effects, and medical history of the patient.

In some embodiments, dosage levels of active ingredients in the pharmaceutical compositions can vary so as to administer an amount of the active compound(s) that can be effective to achieve the desired therapeutic response for a particular patient.

In some embodiments, the selected dose level can depend on the activity of the compound of the present disclosure, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose can be divided into multiple doses for purposes of administration, for example, two to four doses per day. It will be understood, however, that the specific dose level for any particular patient can depend on a variety of factors, including the body weight, general health, diet, time and route of administration and combination with other therapeutic agents and the severity of the condition or disease being treated. The adult human daily dosage can range from between about 1 mg to about 1 g, or from between about 10 mg and 100 mg, of the compound of the present disclosure per 10 kilogram body weight. In some embodiments, a total daily dose can be from 0.1 mg/kg body weight to 100 mg/kg body weight, or from 1 mg/kg body weight to 60 mg/kg body weight, or from 2 mg/kg body weight to 50 mg/kg body weight, or from 3 mg/kg body weight to 30 mg/kg body weight. The daily dose can be administered over one or more administering events over day. In some embodiments, the daily dose can be distributed over two administering events per day (BID), three administering events per day (TID), or four administering events per day (QID). In some embodiments, a single administering event dose ranging from 1 mg/kg body weight to 10 mg/kg body weight is administered BID to a human making a total daily dose from 2 mg/kg body weight to 20 mg/kg body weight. In some embodiments, a single administering event dose ranging from 1 mg/kg body weight to 10 mg/kg body weight is administered TID to a human making a total daily dose from 3 mg/kg body weight to 30 mg/kg body weight. In some embodiments, the amounts of compounds of present disclosure which can be administered to a cell or animal can depend upon numerous factors well understood by one of skill in the art, such as the molecular weights of compounds of present disclosure, and the routes of administration.

In some embodiments, a compound of the present disclosure can be used for treating a number of diseases or conditions for which inhibiting glucosidases is beneficial. Glucosidases are a class of enzymes involved in breaking down complex carbohydrates such as starch and glycogen into their respective monomers. Glucosidases include alpha-amylase, beta-amylase, gamma-amylase, cellulase, sucrase-isomaltase, mannosyl-oligosaccharide glucosidase, alpha-glucosidase, beta-glucosidase, lactase, debranching enzyme, and pullulanase. Alpha-glucosidases include maltase, glucoinvertase, glucosidosucrase, maltase-glucoamylase, alpha-glucopyranosidase, glucosidoinvertase, alpha-D-glucosidase, alpha-glucoside hydrolase, alpha-1,4-glucosidase, and alpha-D-glucoside glycohydrolase.

Exemplary diseases or conditions that can be treated with the compounds and compositions thereof disclosed herein include diabetes, diabetes mellitus type 2, hepatitis C virus (HCV) infection, hepatitis B virus (HBV) infection, dengue virus (DENV) infection, Marburg virus (MARV) infection, Ebola virus (EBOV) infection, BVHV, human immunodeficiency virus (HIV) infection, influenza A infection, influenza B infection, encephalitis virus infection (including, for example, eastern equine encephalitis virus infection (EEEV), western equine encephalitis virus infection (WEEV), Venezuelan equine encephalitis virus infection (VEEV), and Japanese encephalitis virus (JEV) infection), Zika virus infection, yellow fever virus (YFV) infection, Pompe disease, maltase-glucoamylase deficiency, Gaucher disease, mumps, acute pancreatitis, macroamylasemia, sucrase-isolmaltase deficiency, MOGS-CDG, celiac disease, Crohn's disease, and Cori's disease.

In some embodiments, the compounds of the present disclosure can be used for treating a number of diseases or conditions, for which inhibiting ceramide glucosyltransferase and/or lowering a glycosphingolipid concentration is beneficial. Examples of such diseases or conditions include, but are not limited to, Gaucher disease (including Type I, Type II and Type III Gaucher disease), Fabry disease, Sandhoff disease, Tay-Sachs disease, Parkinson's disease, type II diabetes, hypertrophy or hyperplasia associated with diabetic nephropathy, an elevated blood glucose level, and an elevated glycated hemoglobin level, a glomerular disease, and lupus such as systemic lupus erythematosus. Examples of the glomerular disease include, but are not limited to, mesangial proliferative glomerulonephritis, collapsing glomerulopathy, proliferative lupus nephritis, crescentic glomerulonephritis, and membranous nephropathy.

In some embodiments, miglustat can work as a chaperon for mutated acid beta-glucosidase in cells with Gaucher disease mutations. Thus, the compounds of the present disclosure can be used as chaperones.

In some embodiments, a disease or condition, for which inhibiting ceramide glucosyltransferase and/or lowering a glycosphingolipid concentration is beneficial, can be a lysosomal glycosphinglipid storage disease (LSD), such as Gaucher (types I, II and III) disease, Fabry disease, Sandhoff disease, Tay-Sachs disease, GM1 Gangliosidosis, and Niemann-Pick Type C disease.

In some embodiments, a disease or condition, for which inhibiting ceramide glucosyltransferase and/or lowering a glycosphingolipid concentration is beneficial, can be multiple myeloma.

In some embodiments, inhibition of osteoclastogenesis and/or reducing osteoclast activation associated with multiple myeloma with an agent, can be accomplished using one or more of the compounds disclosed herein. In some embodiments, a disease or condition, for which inhibiting ceramide glucosyltransferase and/or lowering a glycosphingolipid concentration is beneficial, can be osteoporosis or osteoarthritis. In some embodiments, inhibition of osteoclastogenesis and/or reducing osteoclast activation associated with these disorders can prevent bone resorption.

In some embodiments, a disease or condition, for which inhibiting ceramide glucosyltransferase and/or lowering a glycosphingolipid concentration is beneficial, can be polycystic kidney disease, such as an autosomal dominant and recessive form of the polycyctic kidney disease. In some embodiments, a disease or condition, for which inhibiting ceramide glucosyltransferase and/or lowering a glycosphingolipid concentration is beneficial, can treat atherosclerosis or renal hypertrophy in a diabetic patient.

In some embodiments, a disease or condition, for which inhibiting ceramide glucosyltransferase and/or lowering a glycosphingolipid concentration is beneficial, can be Type II diabetes, or its related disease or condition. In some embodiments, such disease or condition can be a non-alcoholic fatty liver disease, which can be a consequence of the metabolic syndrome and type II diabetes. In some embodiments, the related disease or condition can be a metabolic syndrome or associated dyslipidemia, which can be a precursor of type II diabetes or atherosclerosis. In some embodiments, the compounds of the present disclosure can be used prophylactically for the prevention of Type II diabetes and/or its related disease or condition.

Without wishing to be bound by a particular theory, the rationale for treating or preventing Type II diabetes and/or its related disease or condition is that compounds of the present disclosure reduce the concentration of glucosylceramide, and can therefore also reduce the expression of gangliosides. Expression of gangliosides, especially GM3, can result in the engagement of insulin receptor into lipid rafts, and cause receptor inactivation and internalization, which results in insulin resistance. Again, without wishing to be bound by a particular theory, it is believed that the compounds of the present disclosure can deplete cellular surfaces of GM3 and sensitize the cells to insulin, thereby being useful in the treatment of insulin resistance. As is well appreciated in the art, insulin resistance can lead to the development of metabolic syndrome, type II diabetes, non-alcoholic liver disease, and atherosclerosis.

In some embodiments, the compounds of the present disclosure can be used for the treatment of a bacterial diseases caused by a toxin, which binds through or to glycosphingolipid or ganglioside. For example, cholera is caused by a toxin (cholera toxin) that binds via its B-subunit to ganglioside GM1. By oral iminosugar treatment of a cholera patient, or by iminosugar treatment of colonic irrigation, the expression of the GM1 target by susceptible cells in the gut epithelium can be abolished or substantially reduced, and have a corresponding therapeutic effect by reducing the effect of the toxin. Another disease involving bacterial toxins can be postdiarrhea hemolytic uremic syndrome, which is commonly associated with particular strains of E. coli bacteria that produce Shiga toxin type-2, which binds to the ganglioside globotriaosylceramide (Gb3). By analogy to the scenario described for cholera therapy, the iminosugars can be used to treat E. coli associated disorders by reducing cellular expression of the ganglioside target of the toxin, such as Gb3. Shiga toxin-2 can be commonly expressed by E. coli 0157:H7 which is a strain of E. coli known to cause enterohemorrhagic disease. The iminosugars can be used therefore to treat enterohemorrhagic disease associated with 0157, and enterohemorrhagic disease caused by other bacteria that express Shiga toxin-2.

In some embodiments, a compound of the present disclosure, such as a compound of Formula (I), Formula (II), Formula (III), Formula (III-A), Formula (III-B), or Formula (III-C), can be administered with one or more additional anti-diabetic drugs.

In some embodiments, a compound of the present disclosure can be administered to an animal in need thereof. Compounds of the present disclosure can inhibit glycosidase function, or they can treat the individual. In one embodiment the treatment can include reducing blood sugar in the animal. Another embodiment includes a method of treating Gaucher's disease and Pompe disease.

In some embodiments, the animal, to whom a compound of the present disclosure can be administered, can be an animal having diabetes, such as a mammal, including, but not limited to, a rodent or a primate, such as a human.

In some embodiments, the amount of a compound of the present disclosure, administered to an animal or to an animal cell to the methods of the invention can be an amount effective to inhibit the glycosidase function. The term “inhibit” as used herein can refer to the detectable reduction and/or elimination of a biological activity exhibited in the absence of a compound of the present disclosure. The term “effective amount” can refer to that amount of any of the compounds described herein necessary to achieve the indicated effect. The term “treatment” as used herein can refer to reducing or alleviating symptoms in a subject, preventing symptoms from worsening or progressing, inhibition or elimination of the causative agent, or prevention of diabetes in a subject who is free therefrom.

In some embodiments, the animal, to whom a compound of the present disclosure can be administered, can be an animal having Type I diabetes, such as a mammal, including, but not limited to, a rodent or a primate, such as a human.

In some embodiments, the animal, to whom a compound of the present disclosure can be administered, can be an animal having Type 2 diabetes, such as a mammal, including, but not limited to, a rodent or a primate, such as a human.

In some embodiment, the animal to whom a compound of the presence disclosure can be administered can be an animal infected with HCV.

In some embodiment, the animal to whom a compound of the presence disclosure can be administered can be an animal infected with HBV.

In some embodiment, the animal to whom a compound of the presence disclosure can be administered can be an animal infected with DENY.

In some embodiment, the animal to whom a compound of the presence disclosure can be administered can be an animal infected with MARV.

In some embodiment, the animal to whom a compound of the presence disclosure can be administered can be an animal infected with EBOV.

In some embodiment, the animal to whom a compound of the presence disclosure can be administered can be an animal infected with BVHV.

In some embodiment, the animal to whom a compound of the presence disclosure can be administered can be an animal infected with HIV.

In some embodiment, the animal to whom a compound of the presence disclosure can be administered can be an animal infected with influenza. In certain embodiments, the influenza can be an influenza A infection or an influenza B infection.

In some embodiment, the animal to whom a compound of the presence disclosure can be administered can be an animal infected with an encephalitis virus, such as western equine encephalitis virus, eastern equine encephalitis virus, Venezuelan equine encephalitis virus, or Japanese encephalitis virus.

In some embodiment, the animal to whom a compound of the presence disclosure can be administered can be an animal infected with Zika virus.

In some embodiment, the animal to whom a compound of the presence disclosure can be administered can be an animal infected with YFV.

The embodiments discussed herein will be further clarified in the following examples. It should be understood that these examples are not limiting to the embodiments described above.

### EXAMPLES

The following Examples are included to demonstrate various aspects of the present disclosure. However, those of skill in the art should, in light of the present disclosure, appreciate that many changes can be made in the specific Example which are disclosed and still obtain a like or similar result without departing from the spirit and scope of the disclosure.

For all chemical scheme Examples given below, intermediate and product identity was confirmed by proton nuclear magnetic resonance spectroscopy (¹H-NMR) and/or liquid chromatography—mass spectrometry (LCMS). Product purity was confirmed by LCMS and high performance liquid chromatography (HPLC) with a target purity of [symbol] approximately 90%. Purity of intermediates was suitable for the intended use. In selected cases (e.g. where the intermediate was considered likely to be labile or where subsequent purification was expected to provide suitable material), the intermediate after workup was moved forward to the next reaction without further purification. References to purification on silica, by column, or by chromatography, unless otherwise specified refer to purification by column chromatography using silica gel (100-200 mesh unless otherwise specified) with the indicated eluent. References to evaporation, or removal or concentration of reaction or volatiles or solvent, unless otherwise specified refer to solvent removal under reduced pressure using a diaphragm vacuum pump and ROTAVAPOR system. References to purification by preparative HPLC unless otherwise specified indicate a KINETEX Evo reverse phase C18 column (5 μm, 250 mm×21.2 mm), with an acetonitrile-water slow gradient and 5 mM ammonium bicarbonate buffer. For some intermediates and products more than one batch was prepared and combined where necessary to provide the desired amounts. Molar equivalents to that of the principal reactant are shown as eq. Reaction endpoints were determined by thin layer chromatography on silica. The Examples below provide representative conditions and scales for single batches. Weights of intermediates and products are approximate. Room temperature (RT) is approximately 20° C. to 35° C.

### Example 1: Synthesis of (1S,2S,3R,4S,5S)-5-(butylamino)-1- (hydroxymethyl)cyclohexane-1,2,3,4-tetraol (Compound 1)

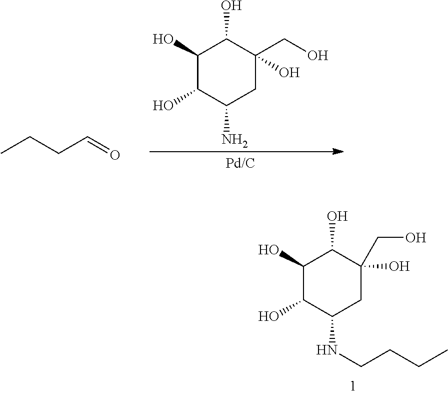

Preparation of Compound 1: Butyraldehyde (200 mg, 2.70 eq) in MeOH (15 mL) was reacted with valiolamine (1.2 eq), a catalytic amount of acetic acid (AcOH), and 10% Pd/C (100 mg), under hydrogen at balloon pressure, at room temperature for 16 hr. The reaction mixture was filtered, and the filtrate was concentrated. Preparative HPLC purification afforded 60 mg of Compound 1 as colorless thick syrup. Proton NMR (500 MHz, CD₃OD): δ 3.79-3.75 (m, 1H), 3.60-3.51 (m, 2H), 3.37-3.35 (m, 2H), 3.30-3.21 (m, 1H), 2.99-2.94 (m, 1H), 2.69-2.63 (m, 1H), 2.08-2.03 (m, 1H), 1.62-1.55 (m, 3H), 1.42-1.35 (m, 2H), 0.89 (t, 3H). Calculated Mass: 249.31. Observed Mass: 250.2 (M+H).

### Example 2: Synthesis of (1S,2S,3R,4S,5S)-5-((6-((4-azido-2- nitrophenyl)amino)hexyl)amino)-1-(hydroxymethyl)cyclohexane-1,2,3,4-tetraol (Compound 2)

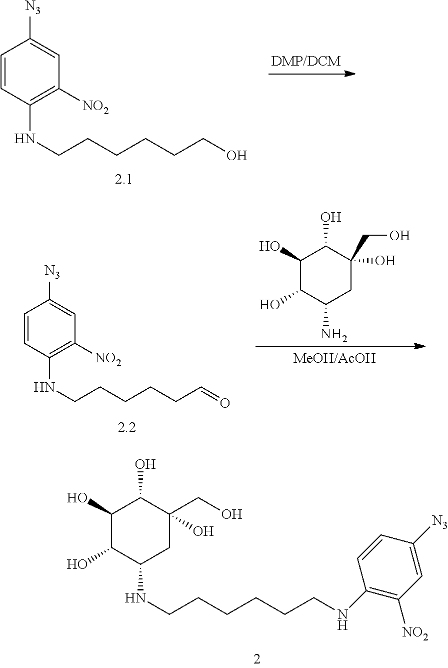 [SMILES: [N-]=[N+]=Nc1ccc(NCCCCCCO)c([N+](=O)[O-])c1 ; [N-]=[N+]=Nc1ccc(NCCCCCC=O)c([N+](=O)[O-])c1 ; N[C@H]1C[C@](O)(CO)[C@@H](O)[C@H](O)[C@H]1O ; [N-]=[N+]=Nc1ccc(NCCCCCCN[C@H]2C[C@](O)(CO)[C@@H](O)[C@H](O)[C@H]2O)c([N+](=O)[O-])c1]

Compound 2.1 can be prepared according to the procedure described in U.S. Pat. No. 8,975,280, the entirety of which is hereby incorporated by reference.

Preparation of 2.2: 2.1 (500 mg, 1.79 mmol) in dichloromethane (DCM, 20 mL) was reacted with Dess Martin Periodinane (DMP, 1.2 eq) at 0° C. The reaction mixture was raised to RT for 2 hr. The reaction was diluted with DCM (20 mL) and washed with saturated sodium bicarbonate (NaHCO₃) solution. The organic layer was dried over anhydrous sodium sulfate (Na₂SO₄), filtered, concentrated, and purified by column chromatography (20% ethyl acetate (EtOAc)/hexane) to afford 350 mg of 2.2.

Preparation of Compound 2: 2.2 (300 mg, 1.00 mmol) in methanol (MeOH, 10 mL) was reacted with valiolamine (0.8 eq), catalytic AcOH, and sodium cyanoborohydride (NaCNBH₃, 1.5 eq) at RT for 16 hr. The volatiles were removed and preparative HPLC purification afforded 106 mg of Compound 2. Proton NMR (400 MHz, CD₃OD): δ7.80 (d, J=2.7 Hz, 1H), 7.26 (dd, J=9.2, 2.8 Hz, 1H), 7.09 (d, J=9.2 Hz, 1H), 3.75-3.67 (m, 1H), 3.57-3.48 (m, 2H), 3.40-3.37 (m, 2H), 3.36 (t, J=1.6 Hz, 1H), 3.12-3.06 (m, 1H), 2.85-2.77 (m, 1H), 2.57-2.45 (m, 1H), 2.01 (dd, J=15.0, 3.2 Hz, 1H), 1.78-1.69 (m, 2H), 1.62-1.39 (m, 8H). Calculated Mass: 454.4. Observed Mass: 455.4 (M+H).

### Example 3: Synthesis of (1S,2S,3R,4S,5S)-1-(hydroxymethyl)-5-((9- methoxynonyl)amino)cyclohexane-1,2,3,4-tetraol (Compound 3)

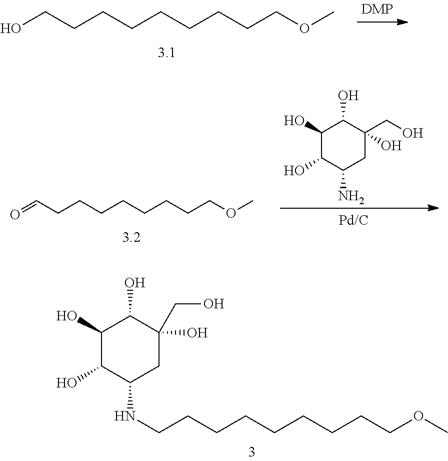 [SMILES: COCCCCCCCCCO ; N[C@H]1C[C@](O)(CO)[C@@H](O)[C@H](O)[C@H]1O ; COCCCCCCCCC=O ; COCCCCCCCCCN[C@H]1C[C@](O)(CO)[C@@H](O)[C@H](O)[C@H]1O]

Methoxynonanol (3.1) can be prepared for example as shown in WO2010096764.

Preparation of 3.2: 3.1 (400 mg, 2.20 mmol) in DCM (25 mL) was mixed with DMP (1.2 eq) at 0° C., then raised to RT and reacted for 4 hr. The reaction was filtered through a celite bed and the filtrate was concentrated. The material was purified by silica gel chromatography (20% EtOAc/hexane) to afford 300 mg of 3.2.

Preparation of Compound 3: 3.2 (300 mg, 1.70 eq) in MeOH (20 mL) was reacted with valiolamine (1.2 eq), catalytic AcOH, and 10% Pd/C (150 mg), under hydrogen gas at balloon pressure, at RT for 16 hr. The reaction was filtered and the filtrate was concentrated. Preparative HPLC purification gave 100 mg of Compound 3 as thick syrup. Proton NMR (500 MHz, CD₃OD): δ 3.75 (t, 1H), 3.59-3.51 (m, 2H), 3.39-3.35 (m, 4H), 3.34-3.31 (m, 4H), 3.10-3.05 (m,1H), 2.80-2.75 (m, 1H), 2.71-2.65 (m, 1H), 2.01 (dd, 1H), 1.49-1.41 (m, 5H), 1.39-1.31 (m, 9H). Calculated Mass: 349.4. Observed Mass: 350.3 (M+H).

### Example 4: Synthesis of (1S,2S,3R,4S, 5S)-1-(hydroxymethyl)-5-((6-((2-nitro-4-(1H- tetrazol-1-yl)phenyl)amino)hexyl)amino)cyclohexane-1,2,3,4-tetraol (Compound 4)

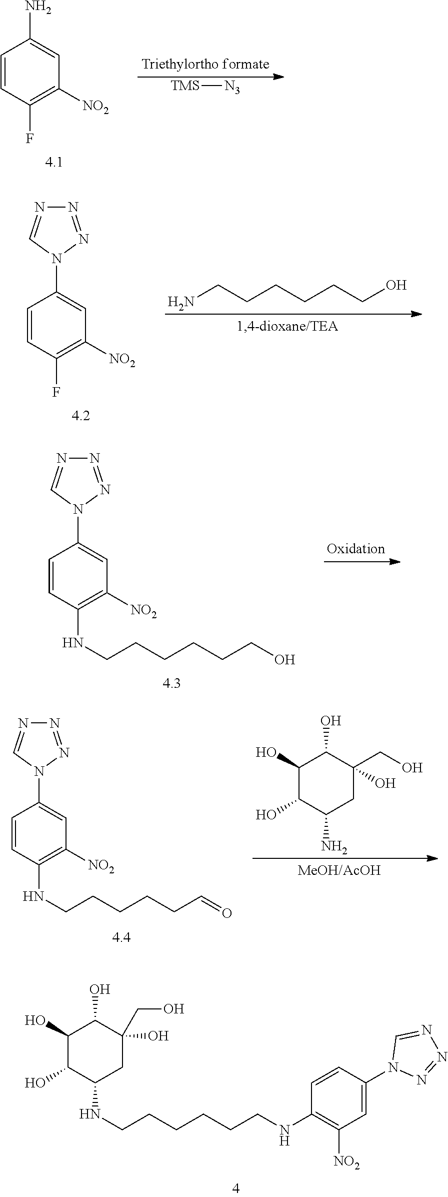

Preparation of 4.2: 4.1 (1.0 g, 6.41 mmol) in acetic acid (20 mL) was mixed with triethylorthoformate (5.0 eq) at 0° C., raised to RT for 30 min, cooled and added with trimethylsilyl azide (TMS-N3) (5.0 eq) at 0° C. The reaction mixture was raised to 80° C. for 4 hr and was concentrated. The residue was diluted with ice-cold water (20 mL) and extracted with EtOAc (2×20 mL). The combined organic layer was washed with water (2×10 mL), dried over anhydrous Na₂SO₄, filtered, and concentrated. The material was purified by silica gel column chromatography (60-120 mesh silica gel) eluting with 20-30% EtOAc/hexane to afford 900 mg of 4.2.

Preparation of 4.3: 4.2 (1.1 g, 5.26 mmol) in 1,4-dioxane (20 mL) was mixed with triethylamine (TEA, 3.0 eq) and aminohexanol (1.2 eq) at RT and was raised to 80° C. for 16 hr. The reaction was concentrated, and the residue was diluted with ice-cold water (30 mL) and extracted with EtOAc (2×50 mL). The combined organic layer was dried over anhydrous Na₂SO₄, filtered, and concentrated. The material was purified by silica gel column chromatography (60-120 mesh silica gel) eluting with 50-100% EtOAc/hexane to afford 1 g of 4.3.

Preparation of 4.4: Oxalylchloride (2.0 eq) in DCM (15 mL) was mixed with dimethyl sulfoxide (DMSO, 4 eq), cooled to −78° C. for 10 min, added with 4.3 (500 mg, 1.63 mmol), and reacted at 78° C. for 20 min. The reaction mixture was quenched with TEA (5.4 eq) at −78° C. for 1 hr, then stirred at RT for 2 hr. Reaction was diluted with ice-cold water and extracted with DCM (2×25 mL). The combined organic layer was dried over Na₂SO₄, filtered and concentrated. The material was purified by silica gel column chromatography (10% EtOAc/hexane) to afford 300 mg of 4.4.

Preparation of Compound 4: 4.4 (300 mg, 1.00 mmol) in MeOH (10 mL) was reacted with valiolamine (0.8 eq), catalytic AcOH, and NaCNBH₃ (1.5 eq) at RT for 16 hr. Volatiles were removed. Preparative HPLC purification afforded 60 mg of Compound 4 as yellow thick syrup. Proton NMR (500 MHz, CD₃OD): δ 9.75 (s, 1H), 8.62 (d, 1H), 7.99-7.90 (m, 1H), 7.25 (d, 1H), 3.79-3.75 (m, 2H), 3.59-3.49 (m, 4H), 3.39-3.35 (m, 2H), 3.10-3.05 (m,1H), 2.80-2.75 (m, 1.6H), 2.71-2.65 (m, 1.4H), 2.01 (dd, 1H), 1.82-1.75 (m, 2H), 1.61-1.43 (m, 6H). Calculated Mass: 481.5. Observed Mass: 482.4 (M+H).

### Example 5: Synthesis of (1S,2S,3R,4S,5S)-1-(hydroxymethyl)-5-((6-((2-nitro-4-(2H- 1,2,3-triazol-2-yl)phenyl)amino)hexyl)amino)cyclohexane-1,2,3,4-tetraol (Compound 5)

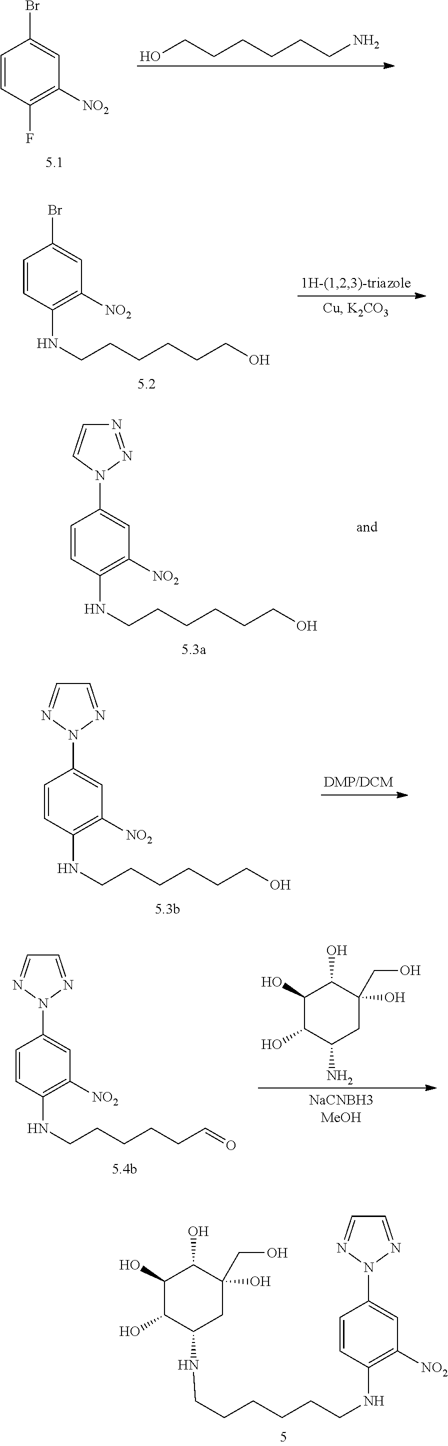

Preparation of 5.2: 4-Bromo-1-fluoro-2-nitrobenzene (10 g, 45.45 mmol) in 1,4-dioxane (60 mL) was mixed with TEA (3.0 eq) and aminohexanol (1.5 eq) at RT. The reaction mixture was raised to 60° C. and reacted for 16 hr. Volatiles were removed and the obtained material was purified by silica gel flash column chromatography (35% EtOAc/hexane) to afford 12 g of 5.2 as orange red solid.

Preparation of 5.3a and 5.3b: 5.2 (5 g, 15.70 mmol) was mixed with 1H-1,2,3-triazole (8.5 eq), Cu (2.1 eq), and K₂CO₃ (1.7 eq) at RT, and raised to 160° C. for 24 hr. Reaction mixture was diluted and mixed with 100 mL EtOAc. The organic layer was separated and concentrated. The mixture of products was purified by silica gel flash column chromatography (30% EtOAc/hexane) to afford 1.6 g of 5.3b as red thick syrup.

Preparation of 5.4b: 5.3b (1.0 g, 3.27 mmol) in DCM (20 mL) was mixed with DMP (1.5 eq) at 0° C., then at RT for 16 hr. The reaction mixture was filtered through a celite bed. The filtrate was concentrated and the material was purified by column chromatography (20% EtOAc/hexane) to afford 300 mg of 5.4b.

Preparation of Compound 5: 5.4b (300 mg, 0.98 mmol) in MeOH (10 mL) was reacted with valiolamine (0.8 eq), catalytic AcOH, and NaCNBH₃ (1.5 eq) at RT for 16 hr. Volatiles were removed and preparative HPLC purification afforded 100 mg of Compound 5 as orange solid. Proton NMR (500 MHz, CD₃OD): δ 8.79 (s, 1H), 8.32-8.21 (m, 1H), 7.91 (s, 2H), 7.21 (dd, 1H), 3.75 (t, 1H), 3.59-3.51 (m, 2H), 3.39-3.35 (m, 2H), 3.34-3.31 (m, 2H), 3.10-3.05 (m, 1H), 2.85-2.80 (m, 1H), 2.59-2.51 (m 1H), 2.01 (dd, 1H), 1.82-1.75 (m, 2H), 1.61-1.42 (m, 7H). Calculated Mass: 480.5. Observed Mass: 481.4 (M+H).

### Example 6: Synthesis of (1S,2S,3R,4S,5S)-1-(hydroxymethyl)-5-((6-((2-nitro-4-(1H- 1,2,3-triazol-1-yl)phenyl)amino)hexyl)amino)cyclohexane-1,2,3,4-tetraol (Compound 6)

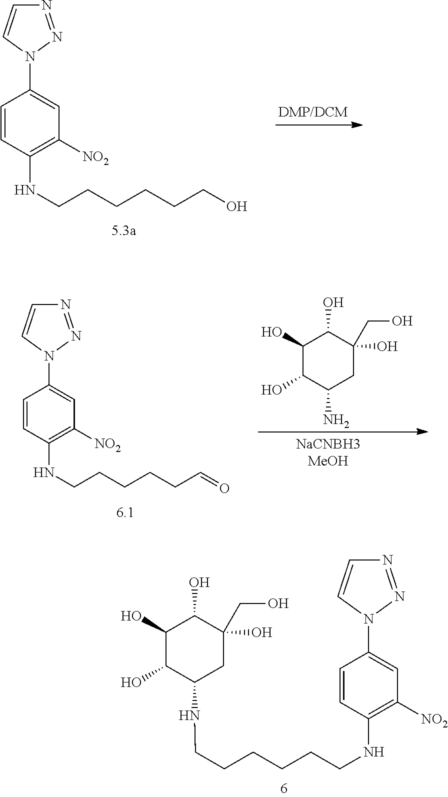 [SMILES: O=[N+]([O-])c1cc(-n2ccnn2)ccc1NCCCCCCO ; O=CCCCCCNc1ccc(-n2ccnn2)cc1[N+](=O)[O-] ; N[C@H]1C[C@](O)(CO)[C@@H](O)[C@H](O)[C@H]1O ; O=[N+]([O-])c1cc(-n2ccnn2)ccc1NCCCCCCN[C@H]1C[C@](O)(CO)[C@@H](O)[C@H](O)[C@H]1O]

Preparation of 6.1: 5.3a (300 mg, 0.98 mmol, from Example 5) in DCM (20 mL) was mixed with DMP (1.5 eq.) at 0° C., then raised to RT for 16 hr. The reaction was filtered through a celite bed. The filtrate was concentrated and the material was purified by column chromatography (40% EtOAc/hexane) to afford 200 mg of 6.1.

Preparation of Compound 6: 6.1 (200 mg, 0.65 mmol) in MeOH (10 mL) was reacted with valiolamine (0.8 eq), catalytic AcOH, and NaCNBH₃ (1.5 eq) at RT for 16 hr. Volatiles were removed and preparative HPLC purification afforded 70 mg of Compound 6 as orange solid. Proton NMR (400 MHz, CD₃OD): δ 8.57 (d, J=2.7 Hz, 1H), 8.49 (d, J=1.0 Hz, 1H), 7.99 (dd, J=9.3, 2.7 Hz, 1H), 7.88 (d, J=1.1 Hz, 1H), 7.24 (d, J=9.4 Hz, 1H), 3.72 (t, J=9.8 Hz, 1H), 3.57-3.43 (m, 4H), 3.40-3.32 (m, 1.93H), 3.12-3.04 (m, 1H), 2.85-2.75 (m, 1.13H), 2.53-2.45 (m, 1.03H), 2.00 (dd, J=14.9, 3.2 Hz, 1.02H), 1.82-1.73 (m, 1.99H), 1.60-1.42 (m, 7.12H). Calculated Mass: 480.5. Observed Mass: 481.1 (M+H).

### Example 7: Synthesis of (1S,2S,3R,4S,5S)-1-(hydroxymethyl)-5-((6-((2-nitro-4- (pyrimidin-2-yl)phenyl)amino)hexyl)amino)cyclohexane-1,2,3 ,4-tetraol (Compound 7)

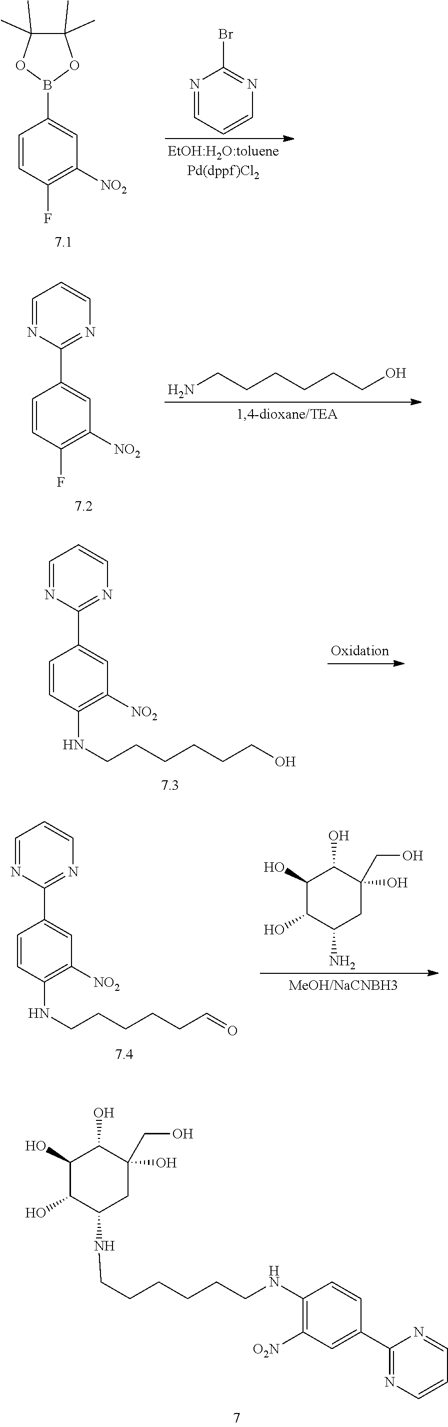

Preparation of 7.4 was carried out as described in WO 2016073652 A1. In brief, 7.1 in EtOH/toluene/water (1:1:1) was coupled to 2-bromopyrimidine (1.2 eq) in the presence of Na₂CO₃ (3.0 eq) and Pd(dppf)₂Cl₂. The reaction was quenched with water and extracted with EtOAc (2×30 mL). The combined organic layer was washed with water, dried over Na₂SO₄, filtered, concentrated and purified by silica chromatography (30% EtOAc/hexane) to afford 7.2. 7.2 was coupled to aminohexanol in 1,4-dioxane and TEA. The reaction was quenched with water and extracted with EtOAc (2×30 mL). The combined organic layer was washed with water, dried over Na₂SO₄, filtered, concentrated and purified by silica chromatography (50% EtOAc/hexane) to afford 7.3. 7.3 (250 mg, 0.70 mmol) in THF (5 mL) was reacted with (COCl)₂ (2.1 eq) and DMSO (2.6 eq) at -78° C. for 2 hr. The reaction mixture was quenched with TEA (5.4 eq) and ice cold water (20 mL), and extracted with DCM (2×30 mL). The combined organic layers were dried over Na₂SO₄, filtered and concentrated to afford 200 mg of 7.4 as colorless thick syrup.

Preparation of Compound 7: 7.4 (200 mg) in MeOH (5 mL) was reacted with valiolamine (0.8 eq), catalytic AcOH and NaCNBH₃ (1.5 eq) at RT for 16 h. The reaction was concentrated, then purified by preparative HPLC to give 200 mg of Compound 7 with 96% HPLC purity as an orange solid. Proton NMR (400 MHz, CD₃OD): δ 9.23 (d, J=2.1 Hz, 1H), 8.78 (d, J=4.9 Hz, 2H), 8.52 (dd, J=9.2, 2.1 Hz, 1H), 7.28 (t, J=4.8 Hz, 1H), 7.14 (d, J=9.2 Hz, 1H), 3.75-3.69 (m, 1H), 3.56-3.50 (m, 2H), 3.49-3.43 (m, 2H), 3.39-3.35 (m, 1H), 3.14-3.08 (m, 1H), 2.92-2.71 (m, 1H), 2.60-2.43 (m, 1H), 2.02 (dd, J=15.0, 3.2 Hz, 1H), 1.82-1.73 (m, 2H), 1.65-1.38 (m, 7H). Calculated Mass: 491.5. Observed Mass: 492.3 (M+H).

### Example 8: Synthesis of (1S,2S,3R,4S,5S)-5-((4-((4-azido-2- nitrophenyl)amino)butyl)amino)-1-(hydroxymethyl)cyclohexane-1,2,3,4-tetraol (Compound 8)

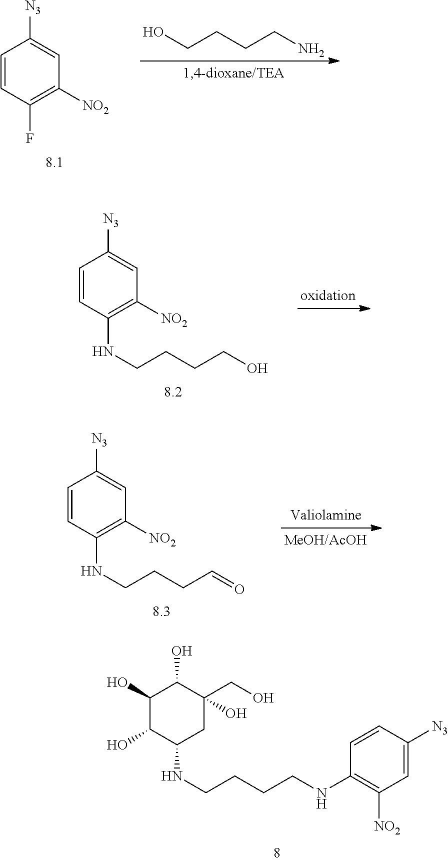 [SMILES: [N-]=[N+]=Nc1ccc(F)c([N+](=O)[O-])c1 ; NCCCCO ; [N-]=[N+]=Nc1ccc(NCCCCO)c([N+](=O)[O-])c1 ; [N-]=[N+]=Nc1ccc(NCCCC=O)c([N+](=O)[O-])c1 ; [N-]=[N+]=Nc1ccc(NCCCCN[C@H]2C[C@](O)(CO)[C@@H](O)[C@H](O)[C@H]2O)c([N+](=O)[O-])c1]

Preparation of 8.2: 8.1 (4-fluoro-3-nitrophenyl azide or 1-fluoro-2-nitro-4-azidobenzene or FNAB) (500 mg, 2.74 mmol) in 1,4-dioxane (10 mL) was reacted with aminobutanol (2.0 eq) and TEA (3 eq) at 80° C. for 16 hr. Reaction was cooled to RT and quenched with ice-cold water. The aqueous layer was extracted with EtOAc (2×20 mL). Combined organic layer was dried over Na₂SO₄, filtered and concentrated. The material was purified by silica chromatography (50% EtOAc/hexane) to afford 600 mg of 8.2 as orange red solid.

Preparation of 8.3: 8.2 (500 mg, 1.99 mmol) in DCM (10 mL) was mixed with DMP (1.5 eq) at 0° C., raised to RT, and reacted for 2 hr. The reaction was quenched with saturated NaHCO₃ solution (10 mL) and extracted with DCM (2×10 mL). The combined organic layer was dried over Na₂SO₄, filtered, concentrated, and purified on silica (30% EtOAc/Hexane) to give 140 mg of 8.3 as orange red solid.

Preparation of Compound 8: 8.3 (140 mg, 0.56 mmol) in MeOH (5 mL) was reacted with valiolamine (0.8 eq), catalytic AcOH and NaCNBH₃ (1.5 eq) at RT for 16 hr. The volatiles were concentrated and purified by preparative HPLC to afford 72 mg of Compound 8 as orange red thick syrup. Proton NMR (400 MHz, CD₃OD): δ 7.81 (d, J=2.8 Hz, 1H), 7.28 (dd, J=9.3, 2.8 Hz, 1H), 7.12 (d, J=9.3 Hz, 1H), 3.78-3.69 (m, 1H), 3.66-3.59 (m, 1H), 3.56 (d, J=10.9 Hz, 1H), 3.49-3.42 (m, 2H), 3.42-3.37 (m, 2H), 3.21-2.99 (m, 2H), 2.95-2.76 (m, 1H), 2.15-2.09 (m, 1H), 1.86-1.72 (m, 4H), 1.71-1.62 (m, 1H). Calculated Mass: 426.4. Observed Mass: 427.1 (M+H), 449.2 (M+Na).

### Example 9: Synthesis of (1S,2S,3R,4S,5S)-5-((5-((4-azido-2- nitrophenyl)amino)pentyl)amino)-1-(hydroxymethyl)cyclohexane-1,2,3,4-tetraol (Compound 9)

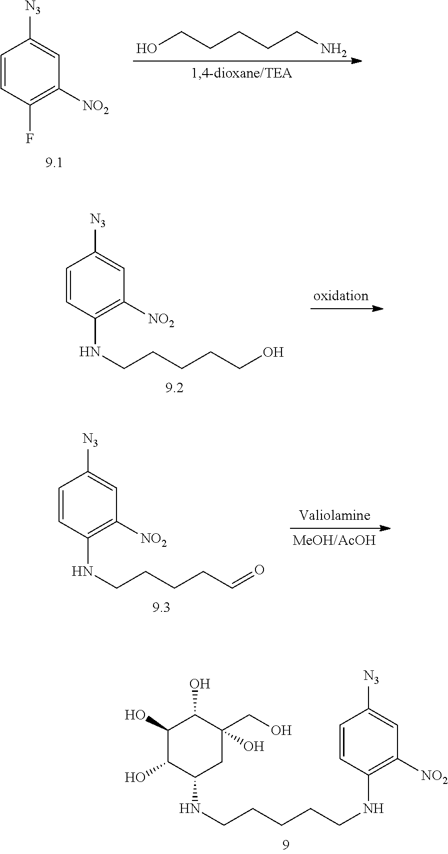 [SMILES: [N-]=[N+]=Nc1ccc(F)c([N+](=O)[O-])c1 ; NCCCCCO ; [N-]=[N+]=Nc1ccc(NCCCCCO)c([N+](=O)[O-])c1 ; [N-]=[N+]=Nc1ccc(NCCCCC=O)c([N+](=O)[O-])c1 ; [N-]=[N+]=Nc1ccc(NCCCCCN[C@H]2C[C@](O)(CO)[C@@H](O)[C@H](O)[C@H]2O)c([N+](=O)[O-])c1]

Preparation of 9.2: 9.1 (FNAB) (1 g, 2.28 mmol) in 1,4-dioxane (20 mL) was mixed with aminopentanol (1.5 eq) and TEA (3 eq) at RT, and raised to 80° C. for 16 hr. The mixture was cooled to RT and the volatiles were removed. The residue was dissolved in water and extracted with EtOAc (2×30 mL). The organic layer was dried over Na₂SO₄, filtered, concentrated, and purified by silica gel column chromatography (20% EtOAc/Hexane) to afford 1.2 g of 9.2 as orange red thick syrup.

Preparation of 9.3: 9.2 (250 mg, 0.94 mmol) in THF (5 mL) was reacted with (COCl)₂ (2.1 eq) and DMSO (2.6 eq) at −78° C. for 2 hr. The reaction was quenched with TEA (5.4 eq) and ice cold water (20 mL), then extracted with DCM (2×30 mL). The combined organic layer was dried over Na₂SO₄, filtered, and concentrated under reduced pressure to afford 180 mg of 9.3 as colorless thick syrup.

Preparation of Compound 9: 9.3 (180 mg) in MeOH (5 mL) was reacted with valiolamine (0.8 eq), catalytic AcOH and NaCNBH₃ (1.5 eq) at RT for 16 hr. The reaction was concentrated to afford 250 mg of material and purified by preparative HPLC to afford 28 mg of Compound 9 as orange red thick syrup with 96% HPLC purity. Proton NMR (400 MHz, CD₃OD): δ 8.11 (br, 1H), 7.81 (d, J=2.8 Hz, 1H), 7.28 (dd, J=9.3, 2.8 Hz, 1H), 7.11 (d, J=9.3 Hz, 1H), 3.81-3.67 (m, 1H), 3.62-3.51 (m, 2H), 3.45-3.34 (m, 4H), 3.27-3.17 (m, 1H), 3.00-2.87 (m, 1H), 2.74-2.59 (m, 1H), 2.11-2.03 (m, 1H), 1.82-1.71 (m, 2H), 1.70-1.46 (m, 5H). Calculated Mass: 440.4. Observed Mass: 441.3 (M+H).

### Example 10: Synthesis of (1S,2S,3R,4S,5S)-5-((2-(2-((4-azido-2- nitrophenyl)amino)ethoxy)ethyl)amino)-1-(hydroxymethyl)cyclohexane-1,2,3,4-tetraol (Compound 10)

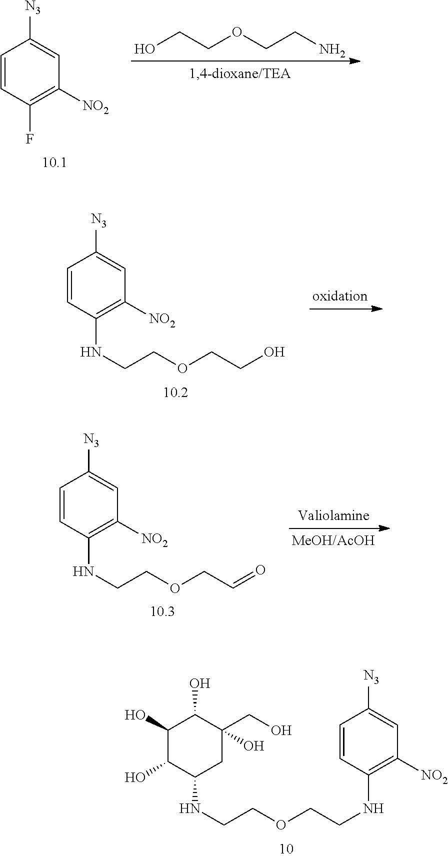 [SMILES: [N-]=[N+]=Nc1ccc(F)c([N+](=O)[O-])c1 ; NCCOCCO ; [N-]=[N+]=Nc1ccc(NCCOCCO)c([N+](=O)[O-])c1 ; [N-]=[N+]=Nc1ccc(NCCOCC=O)c([N+](=O)[O-])c1 ; [N-]=[N+]=Nc1ccc(NCCOCCN[C@H]2C[C@](O)(CO)[C@@H](O)[C@H](O)[C@H]2O)c([N+](=O)[O-])c1]

Preparation of 10.2: 10.1 (FNAB) (500 mg, 2.74 mmol) in 1,4-dioxane (10 mL) was reacted with 2-(2-aminoethoxy)ethan-1-ol (2.0 eq) and TEA (3 eq) at 80° C. for 16 hr. The reaction was cooled to RT and quenched with ice-cold water. The aqueous layer was extracted with EtOAc (2×20 mL). The organic layer was dried over Na₂SO₄, filtered, concentrated, and purified by silica chromatography (50% EtOAc/hexane) to afford 700 mg of 10.2 as orange red solid.

Preparation of 10.3: 10.2 (200 mg, 0.74 mmol) in DCM (50 mL) was reacted with oxalyl chloride (2.1 eq) and DMSO (2.6 eq) at −78° C. for 2 hr. The reaction was quenched with TEA (5.4 eq), then ice cold water (10 mL), and extracted with DCM (2×10 mL). The combined organic layer was dried over Na₂SO₄, filtered and concentrated under reduced pressure. The material was purified by silica gel (30% EtOAc/Hexane) to afford 150 mg of 10.3 as orange red solid.

Preparation of Compound 10: 10.3 (150 mg, 0.56 mmol) in MeOH (7 mL) was reacted with valiolamine (0.8 eq), catalytic AcOH and NaCNBH₃ (1.5 eq) at RT for 16 hr. The volatiles were concentrated under reduced pressure and purified by preparative HPLC to afford 90 mg of Compound 10 as an orange red solid. Proton NMR (400 MHz, CD₃OD): δ 8.24 (br s, 1H), 7.80 (d, J=2.6 Hz, 1H), 7.27 (dd, J=9.3, 2.8 Hz, 1H), 7.14 (d, J=9.3 Hz, 1H), 3.80-3.71 (m, 3H), 3.70-3.63 (m, 1H), 3.63-3.49 (m, 5H), 3.38-3.36 (m, 1H), 3.34 (d, J=4.3 Hz, 1H), 3.13-3.08 (m, 1H), 2.98-2.90 (m, 1H), 2.75-2.67 (m, 1H), 2.01 (dd, J=15.1, 3.3 Hz, 1H), 1.47 (dd, J=2.7, 15.0 Hz, 1H). Calculated Mass: 442.4. Observed Mass: 443.2 (M+H), 465.1 (M+Na).

### Example 11: Synthesis of 5-(((1S,2S,3R,4S,5S)-2,3,4,5-tetrahydroxy-5- (hydroxymethyl)cyclohexyl)amino)pentanoic acid (Compound 11)

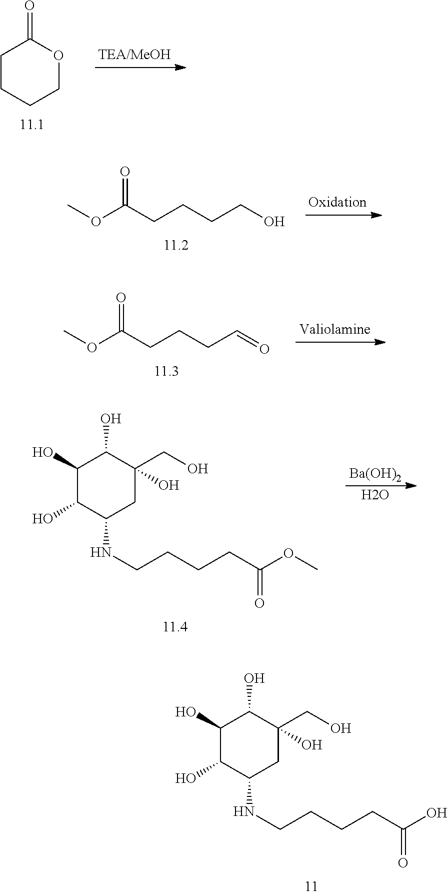

Preparation of 11.2: 11.1 (5 g, 50.0 mmol) in MeOH (50 mL) was reacted with TEA (0.1 eq) at RT for 16 hr. The volatiles were concentrated under reduced pressure. The material was purified by silica gel column chromatography (30% EtOAc/Hexane) to afford 4 g of 11.2 as colorless liquid.

Preparation of 11.3: 11.2 (4 g, 3.37 mmol) in DCM (30 mL) was reacted with (COCl)₂ (2.1 eq) and DMSO (2.6 eq) at −78° C. for 2 hr. The reaction was quenched with TEA (5.4 eq) and ice cold water (20 mL), and extracted with DCM (2×40 mL). The combined organic layer was dried over Na₂SO₄, filtered, and concentrated to afford 1.6 g of 11.3 as colorless thick syrup.

Preparation of 11.4: 11.3 (1.6 g, 12.3 mmol) in MeOH (30 mL) was reacted with valiolamine (0.8 eq), catalytic AcOH, and NaCNBH₃ (1.5 eq) at RT for 16 hr. The volatiles were concentrated and purified by silica gel column chromatography (20% MeOH/DCM, 10% NH₄OH) to afford 800 mg of 11.4 as colorless thick syrup.

Preparation of Compound 11: 11.4 (800 mg, 2.76 mmol) in 15 mL H₂O was mixed with Ba(OH)₂ (2.0 eq) at RT, and increased to 80° C. for 6 hr. The reaction was quenched with dry ice (pH=7) and the obtained solid was filtered through a celite bed and washed with water (10 mL). The filtrate was concentrated and triturated with EtOAc (2×30 mL) then Me0H (2 x 20 mL) followed by lyophilization (3 days) to afford 220 mg of Compound 11 as off-white solid. Proton NMR (400 MHz, D₂O): 63.93-3.77 (m, 2H), 3.68-3.53 (m, 4H), 3.35-3.24 (m, 1H), 3.15-3.04 (m, 1H), 2.35-2.23 (m, 3H), 1.84 (dd, J=16.1, 3.7 Hz, 1H), 1.79-1.57 (m, 4H). Calculated Mass: 293.3. Observed Mass: 294.2 (M+H).

### Example 12: Synthesis of 2-((4-azido-2-nitrophenyl)amino)ethyl (2-(((1S,2S,3R,4S, 5S)- 2,3,4,5 -tetrahydroxy-5 -(hydroxymethyl)cyclohexyl)amino)ethyl)carbamate (Compound 12)

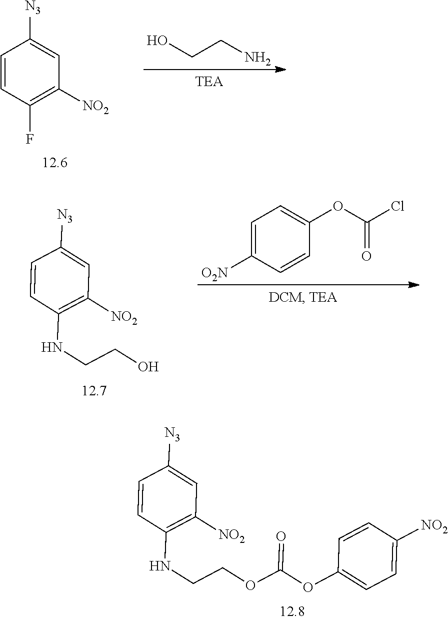

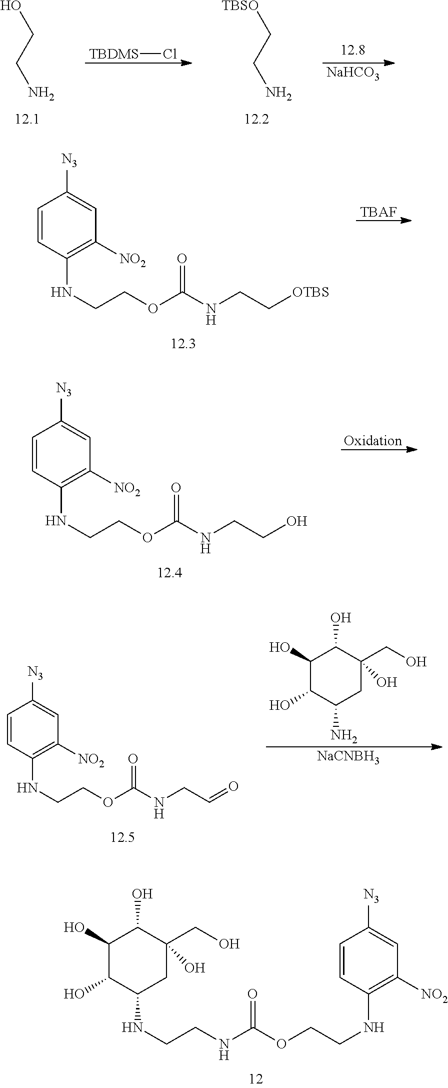

Preparation of 12.2: 12.1 (2-aminoethanol) (5 g, 81.96 mmol) in DCM (120 mL) was reacted with imidazole (2 eq) and TBS-Cl (0.9 eq) at RT for 2 hr. The reaction was diluted with water (400 mL) then extracted with DCM (200 mL×2). Combined organic layer was dried over Na₂SO₄, filtered and concentrated to afford 8 g of 12.2.

Preparation of 12.7: 12.6 (FNAB) (3 g, 16.4 mmol) in 1,4-dioxane (60 mL) was reacted with TEA (3 eq) and aminoethanol (1.5 eq) at 100° C. for 12 hr. The reaction was cooled to RT and the solvent was removed. The residue was dissolved in water (200 mL) and extracted with EtOAc (200 mL×2). The organic layer was dried over Na₂SO₄, filtered and concentrated. The material was purified by silica gel column chromatography (30% EtOAc/hexane) to afford 2 g of 12.7.

Preparation of 12.8: 12.7 (500 mg, 2.24 mmol) in DCM (20 mL) was mixed with TEA (2 eq) and 4-nitrophenyl chloroformate (1.5 eq) at 0° C., and raised to RT for 12 hr. The reaction mixture was quenched with water (20 mL) and extracted with DCM (15 mL×2). The organic layer was dried over Na₂SO₄, filtered and concentrated. The material was crystallized from methanol to afford 600 mg of 12.8.

Preparation of 12.3: 12.8 (600 mg, 1.546 mmol) in THF (20 mL) was reacted with 12.2 (1 eq) and NaHCO₃ (4 eq) at 100° C. in a sealed tube for 12 hr. The reaction mixture was filtered and concentrated. The material was purified by silica gel column chromatography (3% EtOAc/hexane) to afford 400 mg of 12.3.

Preparation of 12.4: 12.3 (400 mg, 0.943 mmol) in THF (10 mL) was reacted with 1.0 M TBAF in THF (1.5 eq) at 0° C. for 2 hr. The reaction was quenched with water (20 mL) and extracted with EtOAc (10 mL×2). The organic layer was dried over Na₂SO₄, filtered and concentrated. The material was purified by silica gel column chromatography (40% EtOAc/hexane) to afford 150 mg of 12.4.

Preparation of 12.5: 12.4 (150 mg, 0.483 mmol) in DCM was reacted with (COCl)₂ (2 eq) and DMSO (4 eq) at −78° C. for 4 h. The reaction was quenched with TEA (6 eq) then water (20 mL), and extracted with DCM (10 mL×2). The organic layer was dried over Na₂SO₄, filtered and concentrated to afford 150 mg of 12.5 used in the next step without further purification.

Preparation of Compound 12: 12.5 (150 mg) in MeOH was reacted with valiolamine (1 eq) and NaCNBH₃ (1.5 eq) at RT for 12 hr. Solvent was removed and preparative HPLC purification afforded 40 mg of Compound 12 with 99% HPLC purity. Proton NMR (400 MHz, CD₃OD): δ 7.81 (d, J=2.6 Hz, 1H), 7.28 (dd, J=9.2, 2.7 Hz, 1H), 7.21-7.13 (m, 1H), 4.37-4.24 (m, 2H), 3.76-3.59 (m, 3H), 3.56-3.43 (m, 2H), 3.37-3.33 (m, 2H), 3.28-3.16 (m, 2H), 3.09-3.06 (m, 1H), 2.90-2.82 (m, 1H), 2.62-2.54 (m, 1H), 2.09-1.86 (m, 1H), 1.57-1.37 (m, 1H). Calculated Mass: 485.4. Observed Mass: 486.4 (M+H).

### Example 13: Glucosidase Assays

Preparation of Human Full-length Glucosidase I: The amino acid sequence for human full-length glucosidase I was obtained from UniProt (Q13724). Constructs were cloned into Gateway entry vector, consisting of the full-length open reading frame (ORF) preceded by a Kozac sequence with a 6-His affinity tag added to the C-terminus (SEQ ID NO 1). The entry vector was cloned into Kemp Protein's BacMam Destination vector and transformed into chemically competent DH10Bac E. coli cells to produce recombinant glucosidase I bacmids. Bacmid isolates were obtained following two rounds of blue-white screening and they were transfected into Sf9 cells cultivated in serum-free medium (Thermo Fisher Scientific). After four days post-transfection, the culture supernatants containing virus were harvested and filter sterilized. The virus titer was determined using a plaque assay on Sf9 cell monolayers and expressions were performed using HEK-293T cells cultivated in serum-free Feestyle-293 medium (Thermo Fisher). A multiplicity of infection (MOI, ratio of virus to cells) of 4 was selected. Soluble glucosidase I was detected by anti-His western blots in the soluble cell extract following extraction with 1% V/V NP40 for 30 minutes at 0° C. Productions and purifications of glucosidase I were carried out at scales ranging from 1-liter to 10-liters. In one example, production at the 1-liter scale was performed in shake-flasks at 27° C. and 100 rpm using HEK-293T cells cultivated in Freestyle-293 medium under serum-free conditions. In another example, production at the 10-liter scale was performed in stirred-tank bioreactors at 27° C. and 80 rpm with dissolved oxygen level at 50% of oxygen in air and pH between 7.0 and 7.2. Cells were transduced at MOI of 4 and the cell pellet was harvested at 48 hours post-transduction using centrifugation.

The pellet from a 10-liter culture was lysed by resuspending the pellet into 1-liter of 50 mM NaH₂PO₄-H₂O, 300 mM NaCl, 10 mM Imidazole, 1 mM PMSF, and 1% V/V NP40 at pH 8. The suspension was incubated on ice for 30 minutes and clarified at 500 g and 4° C. The supernatant was collected and clarified at 20,000 g for 30 minutes at 4° C. and the supernatant was collected and filtered through a 0.2 micron filter. The lysate was loaded onto a 25 mL Ni-NTA Superflow (Qiagen) column (26×60 mm) at a flow rate of 10 mL per minute. The column was washed with 20 column volumes (CV) of 50 mM NaH₂PO₄-H₂O, 300 mM NaCl and 10 mM Imidazole at pH 8 and eluted using a linear gradient of 0-60% of 50 mM NaH₂PO₄-H₂O, 300 mM NaCl and 500 mM Imidazole at pH 8 over 90 minutes at 5 mL per minute. Fractions (25 mL) were collected and analyzed by SDS PAGE and Western Blot (reducing anti-His). Fractions containing purified glucosidase I were pooled and dialyzed into storage buffer (20 mM Tris, 300 mM NaCl, 50 mM L-arginine, 10 mM EDTA and 0.01% V/V Tween 80 pH 7.5). The final material was filter sterilized and stored at 4° C., and the working dilution was confirmed by a use-test in the indicated assay conditions prior to use.

Alpha-glucosidase I Assay: The assay was performed by incubating recombinant alpha-glucosidase I enzyme with multiple dilutions of test compounds for 60 minutes at 37° C. A synthetic trisaccharide substrate analog disclosed by Scaman, C. H., et al., Carbohydrate Research, 296, 203-213 (1996), the entirety of which is hereby incorporated by reference, was then introduced to the mixture for 90 minutes at 37° C. The reaction was then stopped with 1.5 M Tris (pH 8). In the absence of inhibition, the terminal glucose of the substrate was hydrolyzed by the enzyme. The D-glucose product was detected using Amplex Red Glucose/Glucose Oxidase Assay Kit (Invitrogen) following the manufacturer's directions where glucose oxidase reacts with D-glucose to form D-gluconolactone and H₂O₂ which then reacts with the Amplex Red reagent to generate a red-fluorescent oxidation product (560 nm, excitation; 590 nm, emission). Compounds inhibiting glucosidase I inhibit the cleavage of the substrate and result in lower signals. Percent inhibition is plotted as a function of concentration for each compound, compared to control reactions. The IC₅₀ was determined using a 4-PL curve fit and serves as a measure of relative inhibitory activity of each test compound (see Table 4).

Preparation of Murine Full-length Glucosidase II: Glucosidase is a heterodimeric protein consisting of alpha and beta subunits. The amino acid sequence for the alpha (SEQ ID NO: 2) and beta (SEQ ID NO: 3) subunits are full-length murine glucosidase II. A single construct was designed for each of the subunits for gene synthesis and cloning into Gateway entry vectors. The alpha subunit was appended with a Strep-affinity tag and the beta subunit was appended with a 6-His affinity tag. The entry vectors were cloned into Kemp Protein's BacMam Destination vector and the vectors were transformed into chemically competent DH10Bac E. coli cells to produce recombinant glucosidase II bacmids. Bacmid isolates for each of the subunits were obtained following two rounds of blue-white screening and they were transfected into Sf9 cells cultivated in serum-free medium (Thermo Fisher Scientific). MOI of 10 with a ratio of 75% alpha subunit and 25% beta subunit was selected for expression of glucosidase II. Glucosidase II was detected in the culture supernatant and the proteins were detected using Western Blots probed with anti-His and anti-Strep antibodies. Productions and purifications of glucosidase II were carried out at scales ranging from 1-liter to 10-liters. In one example, production at the 1-liter scale was performed in shake-flasks at 27° C. and 100 rpm using HEK-293T cells cultivated in Freestyle-293 medium under serum-free conditions. In another example, production at the 10-liter scale was performed in stirred-tank bioreactors at 27° C. and 80 rpm with dissolved oxygen level maintained at 50% of oxygen in air and pH maintained between 7.0 and 7.2. For both subunits, the cells were transduced using a total MOI of 10 (75% alpha and 25% beta) and the culture supernatant was harvested at 96 hours post-transduction using centrifugation and filtration through a 0.2 micron filter.

The supernatant from a 10-liter culture was placed in a stir-jar and 25 mL of Nickel-SEPHAROSE Excel resin (GE) was added. The supernatant was stirred overnight at 25 rpm and 4° C. and the resin was collected into a column (26 mm×60 mm) and washed with 8 CV of 2X DPBS at pH 7.4. The glucosidase II protein was eluted using a linear gradient of 0-60% of 2X DPBS and 500 mM imidazole at pH 7.4. Fractions (10 mL) were collected and analyzed by SDS PAGE and Western Blot (reducing anti-His and anti-Strep). Fractions containing purified glucosidase II were pooled and concentrated for application to a SUPERDEX 200 (26 mm×600 mm) column (GE). The column buffer was PBS pH 7.2 and the concentrated eluate pool was loaded at a rate of 1 mL per minute. Fractions (3 mL) were collected and analyzed using SDS PAGE and Western Blot (reducing anti-His and anti-Strep). Fractions containing purified glucosidase II in PBS pH 7.2 were pooled and concentrated to 1 mg/mL. The final product was filter sterilized and stored at 4° C.

Alpha-glucosidase II Assay: The assay was performed by incubating recombinant enzyme alpha-glucosidase II for 60 minutes at 37° C. with multiple dilutions of test compounds. 4-methylumbelliferyl-alpha-D-pyranoside was then introduced as the substrate to the mixture. Fluorogenic 1,4-methyllumbelliferone was generated, and the reaction was stopped with the stop solution (0.5 M glycine, 0.3 M NaOH pH 10) after an incubation of 30 minutes at 37° C. 1,4-methyllumbelliferone was detected by fluorescence (excitation at 365 nm, emission at 440 nm). Percent inhibition was then plotted as a function of concentration for each test compound. The 50% inhibitory concentration (IC₅₀) is determined using a 4-PL curve fit and serves as a measure of relative inhibitory activity of each test compound (see Table 4).

Acid alpha-glucosidase (GAA): To analyze inhibitory effects of a test compound, commercially available recombinant human GAA was incubated with multiple dilutions of test compound for 60 minutes at 37° C. 4-methylumbelliferyl-alpha-D-pyranoside was then introduced to the mixture as the substrate. 1,4-methyllumbelliferone is generated, and after an incubation for 20 minutes at 37° C., the reaction was stopped with the stop solution (0.5 M glycine 0.3 M NaOH pH 10). 1,4-methyllumbelliferone is detected by fluorescence (excitation at 365 nm, emission at 440 nm). Relative fluorescence is compared to untreated enzymatic activity and test compound concentration is plotted as a function of percent inhibition. The IC₅₀ is determined using a 4-PL curve fit as the measure of relative inhibitory activity of each test compound (see Table 4).

Beta-glucocerebrosidase (GBA, also referred to glucosylceramidase): To analyze inhibitory effects of a test compound, commercially available recombinant human GBA was incubated with multiple dilutions of test compound for 60 minutes at 37° C. 4-methylumbelliferyl-beta-D-pyranoside was then introduced to the mixture as substrate. Following incubation for 60 minutes at 37° C., 1,4-methyllumbelliferone was generated, and the reaction was stopped with the stop solution (0.5 M glycine; 0.3 M NaOH, pH 10). 1,4-methyllumbelliferone was detected by fluorescence (excitation at 365 nm, emission at 440 nm). Relative fluorescence is compared to untreated enzymatic activity and test compound concentration is then plotted as a function of percent inhibition. The IC₅₀ was determined using a 4-PL curve fit as the measure of relative inhibitory activity of each test compound (see Table 4).

Intestinal alpha-glucosidases (rat maltase and sucrase): Intestinal alpha-glucosidases act upon 1,4-alpha-glucoside bonds, breaking down disaccharides to glucose. To perform this assay, partially purified alpha-glucosidase from intestinal rat powder was incubated for 60 minutes at 37° C. with multiple dilutions of test compounds. Maltose or Sucrose respectively was then introduced as substrate to the mixture and incubated for 30 minutes at 37° C. The reaction was stopped by a 5-minute incubation at 90° C. and the resultant glucose production is detected using Sigma's Glucose GO Kit. Percent inhibition was then plotted as a function of concentration for each test compound. The IC₅₀ was determined using a 4-PL curve fit and serves as the measure of relative inhibition of each test compound (see Table 4). Table 4 summarizes the results of IC50s for each molecule against each tested glucosidase enzyme.

TABLE 4

| Compound | GAA | GBA | sucrase | maltase | aGlc1 | aGlc2 |
|---|---|---|---|---|---|---|
| Valiolamine | 14.83 | >1000 | 0.334005 | 1.298572 | 87.3900 | >>100 |
| Compound 1 | 45.38 | >1000 | 0.070006 | 1.468337 | 29.5400 | 38.69 |
| Compound 2 | 0.839 | 138.6 | <0.0333 | 0.001648 | 0.7300 | 0.0337 |
| Compound 3 | 11.63 | >1000 | 0.116101 | 0.101726 | 10.7700 | 0.4902 |
| Compound 4 | 24.8 | >1000 | 2.928823 | 0.512266 | 32.0500 | 1.419 |
| Compound 5 | 0.514 | 142.9 | 0.034241 | <0.0333 | 0.0479 | <<0.001 |
| Compound 6 | 1.196 | 776.3 | 0.06736 | <0.0333 | 1.2700 | 0.0155 |
| Compound 7 | 0.377 | >1000 | N/A | 0.008144 | 0.6439 | 0.0011 |
| Compound 8 | 0.707 | >1000 | N/A | 0.0053 | 2.4220 | 1.01 |
| Compound 9 | 0.9888 | 102.7 | 0.011174 | 0.004856 | 0.4117 | 0.027 |
| Compound 10 | 8.218 | >1000 | N/A | 0.0261 | 3.6640 | 37.00 |
| Compound 11 | 227.8 | >1000 | 3.163 | 7.576 | 89.8900 | >>100 |
| Compound 12 | N/A | N/A | N/A | N/A | 1.1284 | N/A |

What is claimed is:

1. A compound according to Formula (I): 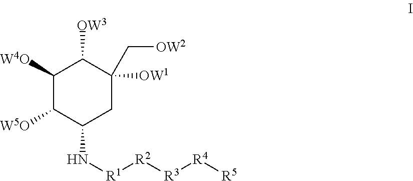
or a pharmaceutically acceptable salt thereof,
wherein
W¹-W⁵ are each independently selected from the group consisting of —H, benzyl, —C(═O)—C₁-C₉ alkyl, and —C(═O)O—C₁-C₉ alkyl;
R¹ is optionally substituted C₁-C₉ alkylene;
R² is absent or selected from the group consisting of —H, —NH—, —O—, —C(═O)—, and —NH—C(═O)O—;
R³ is absent or selected from the group consisting of —O—, —C(═O)—, —C(═O)O—, and optionally substituted C₁-C₆ alkylene;
R⁴ is absent or selected from the group consisting of —H, —NH—, and optionally substituted C₂-C₆ alkylene;
R⁵ is absent, or is 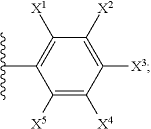
wherein
X¹, X², X³, X⁴, and X⁵ are each independently selected from the group consisting of —H, —NO₂, —N₃, optionally substituted C₂-C₁₂ heterocycle, and optionally substituted C₁-C₁₂ heteroaryl.

2. The compound of claim 1, wherein W¹-W⁵ are each independently -H.

3. The compound of claim 2, wherein R¹ is a C₁-C₉ alkylene.

4. The compound of claim 3, wherein the C₁-C₉ alkylene is selected from the group consisting of —CH₂—, —CH₂—CH₂—, —(CH₂)₃—, —(CH₂)₄—, —(CH₂)₅—, —(CH₂)₆—, —(CH₂)₇—, —(CH₂)₈—, and —(CH₂)₉—.

5. The compound of claim 4, wherein the C₁-C₉ alkylene is —(CH₂)₂—.

6. The compound of claim 4, wherein the C₁-C₉ alkylene is —(CH₂)₄—. 7 The compound of claim 4, wherein the C₁-C₉ alkylene is —(CH₂)₅—.

8. The compound of claim 4, wherein the C₁-C₉ alkylene is —(CH₂)₆—.

9. The compound of claim 4, wherein the C₁-C₉ alkylene is —(CH₂)9—.

10. The compound of claim 1, wherein R³ is a C₁-C₆ alkylene.

11. The compound of claim 10, wherein the C₁-C₆ alkylene is selected from the group consisting of —CH₂—, —CH₂—CH₂—, —(CH₂)₃—, —(CH₂)₄—, —(CH₂)₅—, and —(CH₂)₆—.

12. The compound of claim 11, wherein the C₁-C₆ alkylene is —CH₂—.

13. The compound of claim 11, wherein the C₁-C₆ alkylene is —CH₂—CH₂—.

14. The compound of claim 1, wherein R⁴ is a C₁-C₆ alkylene.

15. The compound of claim 14, wherein the C₁-C₆ alkylene is selected from the group consisting of —CH₂—, —CH₂—CH₂—, —(CH₂)₃—, —(CH₂)₄—, —(CH₂)₅—, and —(CH₂)₆—.

16. The compound of claim 15, wherein the C₁-C₆ alkylene is —CH₂—CH₂—.

17. The compound of claim 1, wherein R⁵ is 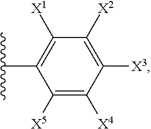
and wherein X¹, X², X³, X⁴, and X⁵ are each independently selected from the group consisting of —H, —NO₂, —N₃, an optionally substituted C₂-C₁₂ heterocycle, and a C₁-C₁₂ heteroaryl.

18. The compound of claim 17, wherein the C₁-C₁₂ heteroaryl is selected from the group consisting of

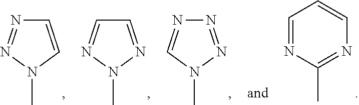

19. The compound of claim 17, wherein X³ is —N₃ or a C₁-C₁₂ heteroaryl.

20. The compound of claim 17, wherein X³ is —N₃.

21. The compound of claim 17, wherein X³ is a C₁-C₁₂ heteroaryl, and wherein the C₁-C₁₂ heteroaryl is selected from the group consisting of

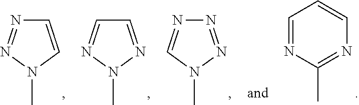

22. The compound of claim 17, wherein X¹ is —NO₂.

23. The compound of claim 17, wherein X², X⁴, and X⁵ are each —H.

24. The compound of claim 1, wherein the compound is selected from the group consisting of:

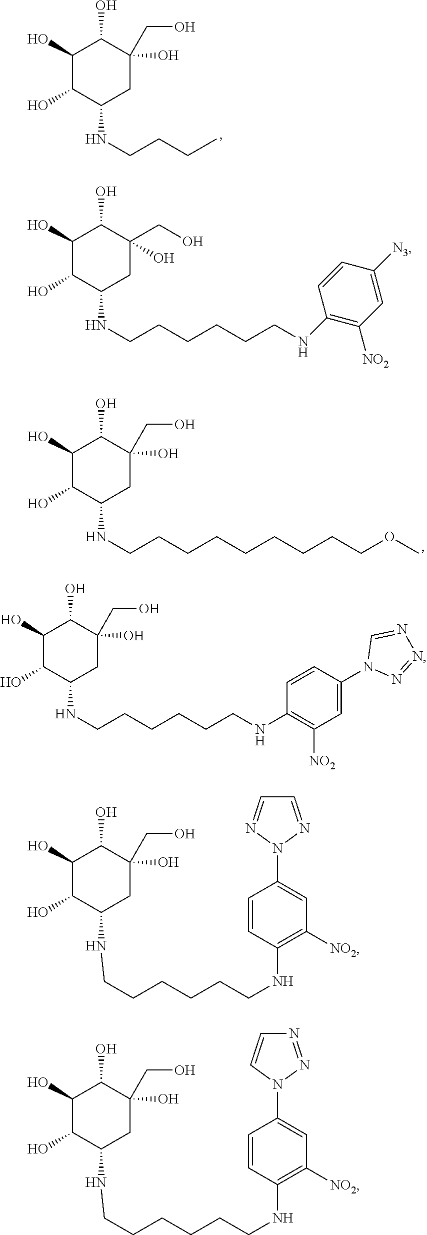

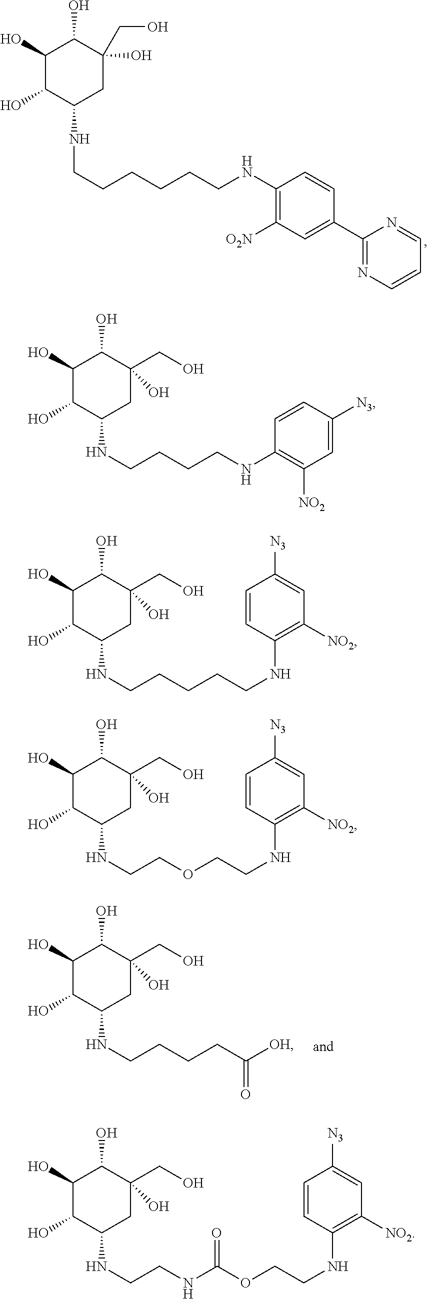

25. A pharmaceutical composition comprising the compound of any one of claims 1-24 and at least one pharmaceutically acceptable excipient.

26. A method of treating diabetes, the method comprising administering to a subject in need thereof, a therapeutically effective amount of the compound of any one of claim 1-24.

27. The method of claim 26, wherein the subject is a mammal.

28. The method of claim 26, wherein the subject is a human.

29. The method of claim 26, wherein the diabetes is Type 1 diabetes.

30. The method of claim 26, wherein the diabetes is Type 2 diabetes.

31. A method for inhibiting glycosidase function, the method comprising administering to a subject in need thereof a therapeutically effective amount of the compound of any one of claims 1-24.

32. A method for treating or preventing a viral infection, the method comprising administering to a subject in need thereof a therapeutically effective amount of the compound of any one of claims 1-24.

33. The method of claim 32, wherein the viral infection is selected from the group consisting of hepatitis C virus (HCV) infection, hepatitis B virus (HBV) infection, dengue virus (DENV) infection, Marburg virus (MARV) infection, Ebola virus (EBOV) infection, BVHV , human immunodeficiency virus (HIV) infection, influenza A infection, influenza B infection, encephalitis viruses infection, Zika virus infection, and yellow fever virus (YFV) infection.

34. The method of claim 33, wherein the encephalitis virus infection is eastern equine encephalitis viruses virus infection, western equine encephalitis virus infection, and Japanese encephalitis virus (JEV) infection.

35. The method of claim 32, wherein the viral infection is dengue virus infection.

36. The method of claim 33, wherein the viral infection is an influenza virus infection.

37. The method of claim 36, wherein the influenza virus infection is an influenza A virus infection.

38. The method of claim 36, wherein the influenza virus infection is an influenza B virus infection.
